(19) United States
(12) Patent Application Publication
(10) Pub. No.: US 2025/0360147 A1
(43) Pub. Date: Nov. 27, 2025

(54) METHODS USING COMBINED ORAL CONTRACEPTIVE COMPOSITIONS WITH REDUCED CARDIOVASCULAR EFFECTS

(71) Applicant: ESTETRA SRL, Liège (BE)

(72) Inventors: Maud JOST, Liège (BE); Glwadys RAUSIN, Liège (BE)

(73) Assignee: ESTETRA SRL, Liège (BE)

(21) Appl. No.: 19/009,804

(22) Filed: Jan. 3, 2025

Related U.S. Application Data
(63) Continuation of application No. 16/573,611, filed on Sep. 17, 2019, now abandoned.
Application No. 16/573,611 is a continuation-in-part of application No. 16/323,110, filed on Feb. 4, 2019, now abandoned.
Application No. 16/323,110 is a continuation-in-part of application No. PCT/EP2019/052980, filed on Feb. 7, 2019.

(30) Foreign Application Priority Data
Aug. 5, 2016 (EP) 16183025.2
Oct. 28, 2016 (WO) PCT/EP2016/076104
Feb. 7, 2018 (EP) 18155571.5
Mar. 7, 2018 (EP) 18160586.6

(51) Int. Cl. A61K 31/565; A61K 9/00; A61K 31/585; A61P 15/18

(52) U.S. Cl. CPC A61K 31/565; A61K 31/585; A61P 15/18; A61K 9/0053

(57) ABSTRACT

Described are combined oral contraceptive compositions with reduced cardiovascular effects and methods using them. In some embodiments, the methods relate to contraceptive methods or methods for treating dysmenorrhea with reduced cardiovascular effects, such as reduced venous thromboembolism (VTE) risk. The methods comprise administering to a female subject an effective amount of an estetrol component and a progestogenic component. The methods enjoy a favourable profile for thromboembolism compared to currently available methods which employs contraceptives from the so-called second, third or fourth generation.

### CROSS-REFERENCE TO RELATED APPLICATIONS

This application is a continuation of U.S. application Ser. No. 16/573,611 filed Sep. 17, 2019, which is a continuation-in-part of U.S. application Ser. No. 16/323,110 filed Feb. 4, 2019, which is the U.S. national stage of PCT/EP2017/069908 (WO 2018/024912) filed Aug. 7, 2017, which claims priority to European Application No. 16183025.2 filed Aug. 5, 2016 and international PCT Application PCT/EP2016/076104 (WO 2018/065076) filed Oct. 28, 2016, and U.S. application Ser. No. 16/573,611 is a continuation-in-part of international PCT Application PCT/EP2019/052980 filed Feb. 7, 2019, which claims priority to European Application 18155571.5 filed Feb. 7, 2018 and European Application 18160586.6 filed Mar. 7, 2018, the entire contents of each of which are incorporated herein by reference.

### FIELD OF THE INVENTION

Described are combined oral contraceptive compositions with reduced cardiovascular effects and methods using them.

In some embodiments, the present invention relates to methods of alleviating the symptoms of dysmenorrhea in a person, comprising administering to said person an effective amount of an estrogenic component. More particularly the estrogenic component is an estetrol component, as further defined herein, and the method enjoys a favourable side-effect profile compared to currently available methods.

In some embodiments, the present invention relates to contraceptive methods with reduced cardiovascular effects, such as reduced thromboembolism risk, such as reduced venous thromboembolism (VTE) risk and reduced aortic thromboembolism (ATE) risk. In some embodiments, the method comprises administering to a female mammal an effective amount for contraception of an estetrol component in combination with a progestogenic component.

As further detailed herein, the methods enjoy a favourable profile for thromboembolism compared to currently available methods, such as a method which employs contraceptives from the so-called second, third or fourth generation.

### BACKGROUND

### Contraception

The first Combined Hormonal Contraceptives (CHCs) contained a dose of estrogen of over 50 μg. However, studies soon showed that these preparations were associated with an unacceptable increase in risk of cardiovascular effects and that this was dependent on the dose of the estrogen component. Subsequent studies showed that the risk was much reduced by lowering the dose of estrogen and this resulted in the introduction of newer preparations containing <50 μg estrogen. However, with the lower doses of estrogen came the realisation that the characteristics of the progestogen may also have an influence on the risk of thromboembolism.

The early progestogens were all derived from testosterone and concerns over their possible cardiovascular risk resulted in the evolution of a newer set of progestogens which were designed with the intention of reducing their adverse cardiovascular impact.

As a result of their phased introduction to the market, the different types of CHCs have been categorised into ‘generations’:
first generation (estrones): refers to the original CHCs that contained estrogens other than ethinylestradiol (EE), and progestogens such as norethisterone and norethindrone;
second generation: (gonanes): refers to the products developed in the 1970s that combined ethinylestradiol with progestogens derived from testosterone (most commonly levonorgestrel (LNG) and norgestrel). These had a higher affinity for the progesterone and androgen receptors compared with the first generation CHCs;
third generation: refers to products developed in the 1980s containing progestogens derived from levonorgestrel (gestodene, desogestrel and norgestimate). It was anticipated these would have less metabolic side effects;
fourth generation: refers to products containing the anti-androgenic progestogens, typically cyproterone acetate, drospirenone (DRSP), dienogest and chlormadinone.

It is important to note that fourth generation COCs have been developed to offer better Quality of Life. The COCs using for example DRSP indeed display an attenuation of a number of side effects compared to earlier generations COCs.

Even though VTE is a rare side effect of all Combined Oral Contraceptives (COCs), it has been shown that:
women using a 3^{rd} generation COC with 30 μg of ethinylestradiol have a small increased risk of VTE compared to women using 2^{nd} generation COCs;
for 3^{rd} generation COCs with 20 μg of ethinylestradiol the epidemiological data do not suggest a lower VTE risk than for those containing 30 μg of ethinylestradiol;
there is an excess risk of VTE during the first year a woman ever uses any COC. The impact of the relative risk of VTE of 3^{rd} generation compared to 2^{nd} generation COCs on the number of additional cases would therefore be greatest in the first year a woman ever uses a COC. This should be taken into account when a COC is prescribed for and used by a woman for the first time.

The estimated incidence of VTE in users of LNG/EE is 10/10,000 women during one year. For users of a contraceptive that contains gestodene/EE, desogestrel/EE or drospirenone/EE the approximate number of cases is 15-20 VTE per 10,000 women during one year.

The estimated risk of blood clot occurrence in users of a CHC based on LNG, norethisterone or norgestimate is from 5 to 7 per 10,000 women during one year. For users of a CHC that contains drospirenone, the approximate risk of blood clot occurrence is from 9 to 12 per 10,000 women during one year. By comparison, the estimated risk of blood clot occurrence in non-CHC users who are not pregnant is around 2 per 10,000 women during one year.

As such, the use of combined contraceptives have been associated with an increased risk in venous thromboembolic events (VTEs). In comparison with non-users, it is generally accepted that the use of second generation COCs multiply by 2 the risk of VTE and that the use of 3^{rd} and 4^{th} generation COCs multiply the risk by 4. The absolute risk of VTE associated with the use of a specific combined contraceptive can only be assessed during very large epidemiological trials. However, and as requested by the European Medicinal Agency, several surrogate markers of the VTE risk can be measured in smaller clinical settings to estimate the risk.

Another way to present the evolution of the venous thrombosis risk as a function of the generation of the COC used is by looking at the relative risk when compared to no oral contraceptive used (relative risk of 1.0). A number of publications (WHO (1995) Lancet 346, 1575-1588; Jick et al. (1995) Lancet 346, 1589-1593; Spitzer et al. (1996) BMJ 312, 127-132; Vlieg et al. (2009) BMJ 339: b2921; Lidegaard et al. (2009) BMJ 339: b2890; Lidegaard et al. (2011) BMJ 343: d6423) have addressed this issue and their findings are summarized in the Table below.

| | Relative Risk |
|---|---|
| No COC | 1.0 |
| 2nd generation COC (levonorgestrel) | 3.2-3.5 |
| 3rd generation COC (desogestrel/gestodene) | 4.8-9.1 |
| 4th generation COC (drospirenone) | 4.0-6.3 |

All the approaches described above relied on COCs employing synthetic estrogens such as ethinyl estradiol (EE), however.

Of particular importance is the fact that estrogens participate in the regulation of the synthesis of a variety of proteins in the liver, such as angiotensinogen, Sex Hormone Binding Globulin (SHBG), ceruloplasmin, Corticosteroid Binding Globulin (CBG), some coagulation factors, coagulation inhibitors or fibrinolysis markers. Changes in these haemostasis markers under the influence of strong estrogens such as EE may collectively contribute to create an imbalance between pro-coagulation and anti-coagulation factors which can enhance the risks of Venous ThromboEmbolism (VTE) events. SHBG plasma levels are a reliable marker of the influence of an estrogen on the synthesis of these proteins by liver cells. This means that a correlation could exist between the level of SHBG induced by a specific COC and the risk of VTE associated with that COC (Odlind V, et al.; Acta Obstet Gynecol Scand 2002; 81:482).

Although cohort studies performed on a sufficient number of subjects are required to evaluate the risk of VTE with a specific COC, different haemostatic markers and carrier proteins (such as SHBG) can be measured to estimate this risk on a limited number of subjects.

On the effect of the progestogenic component, a substantial number of studies plus two good meta-analyses have now evaluated the thrombotic risk with drospirenone containing CHCs. These use a number of different data sources across different countries and, with the exception the study by Dinger (Dinger J, Assmann A, Mohner S, Minh T D. Risk of venous thromboembolism and the use of dienogest- and drospirenone-containing oral contraceptives: results from a German case-control study. J Fam Plann Reprod Health Care. 2010; 36 (3): 123-9), all consistently show an elevated risk of VTE in drospirenone users relative to levonorgestrel users that was, in most cases, statistically significant. The risk estimates most commonly range between about 1.5 and 2 times versus levonorgestrel. While limitations can always be identified for observational studies, bias and residual confounding are unlikely to account for the entire risk increase that is observed. The Sidney study (Sidney S, Cheetham T C, Connell F A, Quellet-Hellstrom R, Graham D J, Davis D, Sorel M, Quesenberry C P Jr, Cooper W O. Recent combined hormonal contraceptives (CHCs) and the risk of thromboembolism and other cardiovascular events in new users. Contraception 2013, 87 (1): 93-100) in particular is considered to provide strong evidence as this analysis was restricted to new users (of which there were almost 140,000 in this cohort study).

Overall, consistent findings support an excess VTE risk with DRSP in relation to LNG.

There thus remains a need for a contraceptive approach which provides a better safety profile than currently available COCs, and in particular which displays a lower risk of thromboembolic events. This is especially critical in at-risk populations, such as first-ever users; switchers/re-starters with a break of >4 weeks, including a break of three months; and women with increased baseline risk for VTE due to one or more major risk factors (such as, but not limited to, BMI>30, older age, and positive personal and/or family history).

### Treating Dysmenorrhea

Nonsteroidal anti-inflammatory drugs (NSAIDs) are considered the first line of therapy (Proctor M, Farquhar C; Clin Evid 2003; 1994—Zhang W Y, Li Wan Po A.; Br J Obstet Gynaecol 1998; 105:780-French L.; Am Fam Physician 2005; 71:285). NSAIDs should be started at the onset of menses and continued for the first one to two days of the menstrual cycle or for the usual duration of crampy pain. Patients with severe symptoms should begin taking NSAIDs one to two days prior to the onset of menses.

Combined Oral Contraceptive pills (COCs) can be given to patients who fail to respond to or cannot tolerate NSAIDs (Davis A R, et al.; Obstet Gynecol 2005; 106:97). COCs prevent menstrual pain by suppressing ovulation, thereby decreasing uterine prostaglandin levels. An additional mechanism may result from the reduction of menstrual flow after several months of use. In a sexually active female, COCs may be considered for first-line of therapy because they serve a dual purpose: prevention of both pregnancy and dysmenorrhea.

In view of the issues discussed above, there remains a need for a therapeutic approach to dysmenorrhea which, on the one hand, has as little side effects as possible, but on the other hand proves very efficient in the management of dysmenorrhea.

### SUMMARY

In accordance with some aspects, the present invention relates to contraceptive methods and methods for treating dysmenorrhea with reduced cardiovascular effects, comprising administering to a female mammal an effective amount of an estetrol component in combination with a progestogenic component.

In some embodiments, the number, frequency and/or severity of VTE events is reduced, compared to other contraceptive methods or other methods for treating dysmenorrhea.

In some embodiments, the risk of VTE during a contraceptive method as described herein is similar to the VTE risk during use of a CHC based on levonorgestrel, norgestimate or norethisterone.

In some embodiments, the risk of VTE during a contraceptive method as described herein is lower than the VTE risk during use of a CHC based on levonorgestrel, norgestimate or norethisterone.

In some embodiments, the number, frequency and/or severity of pulmonary embolism events is reduced during a contraceptive method as described herein, compared to other contraceptive methods.

In some embodiments, the number, frequency and/or severity of deep venous thrombosis (DVT) events during a contraceptive method as described herein is reduced, compared to other contraceptive methods.

In some embodiments, no haemostatic change that exceeds the boundaries of the normal range, as further defined herein, occurs upon administration of the compositions described herein, for the methods described herein.

In some embodiments, the method is associated with smaller “haemostatic changes” (as further defined herein) than a contraceptive method employing a 2^{nd}, 3^{rd} or 4^{th} generation CHC. In some embodiments, the method is associated with smaller “haemostatic changes” than a contraceptive method employing a 2^{nd} generation CHC.

In some embodiments, markers of coagulation and/or fibrinolysis exhibit smaller changes (by comparison with their levels before administration of any contraceptive composition) during a contraceptive method as described herein as compared to changes observed with another contraceptive method.

In some embodiments, markers of coagulation and/or fibrinolysis markers exhibit smaller changes during a contraceptive method as described herein than during a contraceptive method using a 2^{nd}, 3^{rd} or 4^{th} generation CHC. In some embodiments, markers of coagulation and/or fibrinolysis markers exhibit smaller changes during a contraceptive method as described herein than during a contraceptive method using a 2^{nd} generation CHC.

In some embodiments, an effective amount of an estetrol component is used in a combined oral contraceptive composition which uses drospirenone as the progestogenic component, and is effective to reduce the risk of blood clots associated with the use of drospirenone.

In some embodiments, an effective amount of an estetrol component is used in a combined oral contraceptive composition which uses drospirenone as the progestogenic component, and is effective to reduce the risk of blood clots associated with the use of a combined oral contraceptive composition which includes drospirenone.

In any of these aforementioned embodiments, the risk of blood clots may be advantageously reduced to less than 8, less than 7, less than 6, less than 5, or less than 4 per 10,000 women using said contraceptive during one year.

In embodiments relating to the treatment of dysmenorrhea, there are provided methods of alleviating the symptoms of dysmenorrhea in a person, comprising administering to said person an effective amount of an estetrol component, as further defined herein, wherein the method enjoys a favourable side-effect profile compared to currently available methods. In some embodiments, one or more of the number, the frequency and the severity of treatment-related side effects is reduced, compared to other dysmenorrhea treatments of similar efficacy. In some embodiments, the number, frequency and/or severity of headaches is reduced, compared to other dysmenorrhea treatments of similar efficacy. In some embodiments, the number, frequency and/or severity of breast pain events is reduced, compared to other dysmenorrhea treatments of similar efficacy.

In some embodiments, the method involves the administration of an effective amount for contraception or for treating dysmenorrhea of an estetrol component and of a progestogenic component.

In some embodiments, the estetrol and the progestogenic components are included in a single dosage unit. In further embodiments, the dosage unit is a daily dosage unit.

For contraception or for treating dysmenorrhea, the progestogenic component may be drospirenone and may be used at a daily dose of from 0.5 mg to 10 mg, or at a daily dose of from 1 mg to 4 mg.

For contraception or for treating dysmenorrhea the estetrol component may be used at a daily dose of from 1 mg to 40 mg, or at a daily dose of from 5 mg to 25 mg, or at a daily dose of from 10 mg to 20 mg. In particular embodiments, the estetrol component is estetrol monohydrate.

In specific embodiments, the estetrol component is estetrol monohydrate and is used at a daily dose of about 15 mg and the progestogenic component is drospirenone and is used at a daily dose of about 3 mg.

In the following numbered paragraphs, additional embodiments are described.
1. A contraceptive method for a female mammal, which comprises administration of a composition comprising an effective amount of an estetrol component.
2. The contraceptive method of paragraph 1 wherein said composition further comprises a progestogenic component.
3. The contraceptive method of paragraph 1 or 2 wherein said method results in the reduction of one or more of the number, the frequency and the severity of VTE events compared to other contraceptive methods.
4. The contraceptive method of paragraph 3 wherein said progestogenic component is drospirenone.
5. A contraceptive method according to any one of paragraphs 1˜4 wherein the estetrol component is used at a daily dose of from 1 mg to 40 mg, or at a daily dose of from 5 mg to 25 mg.
6. The contraceptive method of paragraph 4 wherein drospirenone is used at a daily dose of from 0.5 mg to 10 mg, or at a daily dose of from 1 mg to 4 mg.
7. A contraceptive method according to any one of paragraphs 1-6 wherein the administration method is a combined method with an administration-free interval of about 7 days, or with an administration-free interval of about 4 days.
8. A contraceptive method according to any one of paragraphs 1-7 wherein the estetrol component is estetrol, or estetrol monohydrate.
9. The contraceptive method according to paragraph 8 wherein the estetrol component is used at a daily dose of about 15 mg of estetrol.
10. The contraceptive method according to paragraph 9 wherein the drospirenone is used at a daily dose of about 3 mg.
11. A contraceptive method according to any of the preceding paragraphs wherein the composition is formulated as an oral dosage unit.
12. The contraceptive method according to paragraph 11 wherein the oral dosage unit is formulated to correspond to a daily dosage unit.
13. A contraceptive method according to any one of paragraphs 3-12 wherein the risk of blood clots occurrence is less than 12, less than 11, less than 10, or less than 9 per 10,000 women using said composition during one year.
14. A contraceptive method according to paragraph 13 wherein the risk of blood clots occurrence is less than 8, less than 7, less than 6, less than 5, or less than 4 per 10,000 women using said composition during one year.
15. A contraceptive method according to any one of the paragraphs 1-12 wherein the number of VTE cases is less than 15-20 VTE per 10,000 women during one year.
16. A contraceptive method according to paragraph 15 wherein the number of VTE cases is less than 15, less than 13, less than 11, or less than 10 VTE per 10,000 women during one year.
17. A contraceptive method according to any one of the preceding paragraphs wherein the VTE risk is lower than the VTE risk associated with a 3^{rd} generation or with a 4^{th} generation COC.
18. A contraceptive method according to any one of the preceding paragraphs wherein the VTE risk is lower than the VTE risk associated with a 2^{nd} generation COC.
19. A contraceptive method according to paragraph 17 or 18 wherein the profile of side effects other than VTE is better than the profile of side effects associated with a 2^{nd} generation COC.
20. A contraceptive method according to any one of the preceding paragraphs wherein the relative venous thrombosis risk, by comparison to a woman not using COCs, is lower than 4.5, lower than 4.0, lower than 3.5, lower than 3.2, or lower than 3.0.
21. A contraceptive method according to any one of the preceding paragraphs wherein the percentage of subjects in the high risk category for thrombosis, defined as subjects having an odds ratio of 2 or above for thrombosis risk compared to subjects not using COCs, represents less than 40%, less than 35%, less than 30%, less than 25%, or less than 20%, of the total population using said composition.
22. A contraceptive method according to any one of the preceding paragraphs wherein the composition is administered to a woman who is a first-ever user, or a woman who is a switcher or a re-starter with a break of >4 weeks (including three months or longer), or a woman with increased baseline risk for VTE due to one or more major risk factors selected from BMI>30, older age, and positive personal and/or family history.

In the following numbered paragraphs, additional embodiments are described.
23. A contraceptive method for a female mammal, which comprises administration of a combined oral contraceptive comprising an effective amount of an estetrol component as the estrogenic component and an effective amount of drospirenone as the progestogenic component, wherein the estetrol component reduces the risk of thromboembolism associated with the use of drospirenone.
24. The contraceptive method of paragraph 23, wherein said risk of thromboembolism is reduced to less than 8, less than 7, less than 6, less than 5, or less than 4 per 10,000 women using said contraceptive during one year.
25. The contraceptive method of paragraphs 23 or 24, wherein the estetrol component is used at a daily dose of from 1 mg to 40 mg, or at a daily dose of from 5 mg to 25 mg.
26. The contraceptive method of any of paragraphs 23-25 wherein drospirenone is used at a daily dose of from 0.5 mg to 10 mg, or at a daily dose of from 1 mg to 4 mg.
27. The contraceptive method of any one of paragraphs 23-26 wherein the administration method is a combined method with an administration-free interval of about 7 days, or with an administration-free interval of about 4 days.
28. The contraceptive method of any one of paragraphs 23-27 wherein the estetrol component is estetrol, or estetrol monohydrate.
29. The contraceptive method of paragraph 28 wherein estetrol is used at a daily dose of about 15 mg of estetrol.
30. The contraceptive method of paragraph 29 wherein drospirenone is used at a daily dose of about 3 mg.
31. The contraceptive method of any of the preceding paragraphs wherein the contraceptive is formulated as an oral dosage unit.
32. The contraceptive method according to paragraph 31 wherein the oral dosage unit is formulated to correspond to a daily dosage unit.
33. The contraceptive method according to any one of the preceding paragraphs wherein the VTE risk is lower than the VTE risk associated with a 2^{nd} generation COC.
34. The contraceptive method according to paragraphs 32 or 33 wherein the profile of side effects other than VTE is better than the profile of side effects associated with a 2^{nd} generation COC.
35. The contraceptive method according to any one of the preceding paragraphs wherein the relative venous thrombosis risk, by comparison to a woman not using COCs, is lower than 3.5, lower than 3.2, lower than 3.0, lower than 2.5, or lower than 2.0.
36. The contraceptive method according to any one of the preceding paragraphs wherein the percentage of subjects in the high risk category for thrombosis, defined as subjects having an odds ratio of 2 or above for thrombosis risk compared to subjects not using COCs, represents less than 40%, less than 35%, less than 30%, less than 25%, or less than 20%, of the total population using said composition.
37. The contraceptive method according to any one of the preceding paragraphs wherein the composition is administered to a woman who is a first-ever user, or a woman who is a switcher or a re-starter with a break of >4 weeks (including three months or longer), or a woman with increased baseline risk for VTE due to one or more major risk factors selected from BMI>30, older age, and positive personal and/or family history.

### BRIEF DESCRIPTION OF THE DRAWINGS

FIGS. 1A to IF present the changes observed in 20 haemostasis parameters at Cycle 6. For each parameter, the mean relative change from baseline to Cycle 6, in percentage, is displayed for the E4/DRSP group (black bars), for the EE/LNG group (oblique lines) and for the EE/DRSP group (horizontal lines). The data also appears in Table 3 in Example 5 below. FIG. 1A presents five parameters which are coagulation factors. FIG. 1B presents five parameters which are anticoagulant proteins. FIG. 1C presents four parameters which relate to functional clotting tests. FIG. 1D presents three parameters which are fibrinolysis proteins. FIG. 1E presents two parameters which are markers of ongoing coagulation. And FIG. 1F presents the levels observed for SHBG.

FIG. 2 displays the APC-r change (ETP) between Cycle 6 and baseline. The data also appears in Table 1 in Example 5 below.

FIG. 3 displays the change of D-Dimer and prothrombin fragment 1+2 between Cycle 6 and baseline. The data also appears in Table 1 in Example 5 below.

### DETAILED DESCRIPTION

### Definitions

The term “estetrol component”, as used throughout this document, encompasses substances selected from the group consisting of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms; and combinations thereof. In some embodiments, the estetrol component is estetrol (including estetrol hydrates). In some embodiments, the estetrol component contained in the dosage unit is estetrol monohydrate.

The term “progestogenic component” is defined as a substance that is capable of triggering a progestogenic response in vivo or a precursor which is capable of liberating such a substance in vivo. Usually progestogenic components are capable of binding to a progestogen receptor.

“About” as used herein referring to a measurable value such as a parameter, an amount, a temporal duration, and the like, is meant to encompass variations of +/−10% or less, more preferably +/−5% or less, even more preferably +/−1% or less of and from the specified value, in so far such variations are appropriate to perform in the disclosed invention. However, it is to be understood that the value to which the modifier “about” refers is itself also specifically disclosed.

The term “an effective amount” refers to an amount necessary to obtain a physiological effect. The physiological effect may be achieved by one dose or by repeated doses.

In the context of contraception “an effective amount” refers to an amount which is effective to suppress ovulation.

In the context of treating dysmenorrhea “an effective amount” refers to an amount which is effective in reducing, eliminating, treating or controlling the symptoms of dysmenorrhea. The term “controlling” is intended to refer to all processes wherein there may be a slowing, interrupting, arresting, or stopping of the progression of dysmenorrhea, but does not necessarily indicate a total elimination of dysmenorrhea, and is intended to include prophylactic treatment and chronic use.

As used herein, the terms ‘blood clot risk”, are taken as equivalent to “thromboembolism risk” and to “venous thromboembolism risk”.

The terms “2^{nd} generation COC”, as used herein, refers to a COC combining ethinylestradiol as the estrogen with levonorgestrel (LNG) or norgestrel as the progestogenic component.

The terms “3^{rd} generation COC”, as used herein, refers to a COC using gestodene, desogestrel or norgestimate as the progestogenic component.

The terms “4^{th} generation COC”, as used herein, refers to a COC using cyproterone acetate, drospirenone, dienogest, nomegestrol or chlormadinone as the progestogenic component.

As used herein, “positive family history”, when used in the context of risk factors, refers to family history of venous or arterial thromboembolism or pulmonary embolism, known thrombogenic mutations (by way of example, but not limitation, Factor V Leiden; prothrombin mutation; protein S, protein C and antithrombin deficiencies) which are considered as risk factors for venous thromboembolism in combined oral contraceptive users.

As used herein, “positive personal history”, when used in the context of risk factors, refers to personal history of venous or arterial thromboembolism or pulmonary embolism, which are considered as a risk factor for venous thromboembolism in combined oral contraceptive users.

As used herein, “older age”, when used in the context of risk factors, refers to an age over 35 years.

As used herein a woman is a “first-ever user”, when she is in her first year of ever using CHC. This is generally understood to be a period of increased VTE risk.

As used herein a woman is a “re-starter” with a break of >4 weeks when she has discontinued use of CHC for 4 weeks or more, including a break of three months or longer. There is some evidence that the VTE risk is increased when CHC is re-started after a break in use of 4 weeks or more.

As used herein a woman is a “switcher” when she discontinues the use of one type of CHC to initiate the use of another type of CHC. Optionally, this switch comprises a break in use of 4 weeks or more.

As used herein, “BMI” stands for Body Mass Index, and is defined as the body mass divided by the square of the body height, and is expressed in units of kg/m², resulting from mass in kilograms and height in metres.

As used herein, “COC” stands for Combined Oral Contraceptive and “CHC” stands for Combined Hormonal Contraceptive. Throughout this patent application these two terms are used interchangeably, such that embodiments describing COC(s) should be read as embodiments describing CHC(s) and vice-versa. In particular, 2^{nd}, 3^{rd} and 4^{th} generation COCs are equivalent to 2^{nd}, 3^{rd} and 4^{th} generation CHCs, respectively.

As illustrated in the Example, the present contraceptive methods have proved to have a surprisingly safe profile, especially by direct comparison to contraceptive methods employing either 4^{th} generation or 2^{nd} generation COCs.

The present method employs an estetrol component which is a natural estrogen (i.e. found in nature) and a biogenic estrogen (i.e. occurring naturally in the human body). Because biogenic estrogens are naturally present in the fetal and female body, a good tolerability and safety profile are observed, particularly if the serum levels resulting from the exogenous administration of such estrogens do not substantially exceed naturally occurring concentrations.

Another important benefit of the present estetrol component is derived from its relative insensitivity to interactions with other drugs (drug-drug interactions). It is well known that certain drugs may decrease the effectiveness of estrogens, such as ethinyl estradiol, and other drugs may enhance their activity, resulting in possible increased side-effects. Similarly, estrogens may interfere with the metabolism of other drugs. In general, the effect of other drugs on estrogens is due to interference with the absorption, metabolism or excretion of these estrogens, whereas the effect of estrogens on other drugs is due to competition for metabolic pathways.

The clinically most significant group of estrogen-drug interactions occurs with drugs that may induce hepatic microsomal enzymes which may decrease estrogen plasma levels below therapeutic level (for example, anticonvulsant agents; phenytoin, primidone, barbiturates, carbamazepine, ethosuximide, and methosuximide; antituberculous drugs such as rifampin; antifungal drugs such as griseofulvin). The present estrogenic substances are not dependent on up- and downregulation of microsomal liver enzymes (e.g. P450's) and also are not sensitive to competition with other P450 substrates. Similarly, they do not interfere significantly in the metabolism of other drugs.

In particular, estetrol at a high concentration of 10 μmol/l does not inhibit (less than 10%) the major cytochrome P450 enzymes (CYP1A2, CYP2C9, CYP2C19, CYP2D6 and CYP3A4) unlike estradiol. Indeed, estradiol exerts a substantial inhibitory effect on CYP2C19 and CYP1A2 of 63% and 19%, respectively. Similarly, ethinyl estradiol, which is the estrogen used in a large number of COCs, exerts a substantial inhibitory effect on CYP2C19 and CYP3A4 of 82% and 45%, respectively.

The above observations serve to explain why the estetrol component of the invention hardly suffer from drug-drug interactions and thus produce a very consistent, i.e. predictable, impact. Thus, the efficacy of the estetrol substances of the invention is highly reliable.

Additionally, the terminal half-life of the naturally occurring estrogens ranges from 2 to 14 hours while estetrol is characterized by a terminal half-life of 31.7 hours. Consequently, the use of estetrol in the method of the invention allows for a more than 24-hour coverage of the receptors by the treatment. This pharmacokinetic property enhances the efficacy of the product even in case of low treatment compliance by the user.

It has to be noted that when estetrol (E4) is associated with 3 mg drospirenone (DRSP), the bleeding profile and the cycle control is improved in comparison to other combined oral contraceptives using a physiological estrogen, namely estradiol-valerate (E2V) or estradiol (E2).

In a study evaluating the bleeding pattern and cycle control of different E4/DRSP or E4/LNG combinations in comparison to a marketed quadriphasic combined oral contraceptive containing E2V and desogestrel (DSG), the combination of 15 mg E4/DRSP and the combination 20 mg E4/LNG were both associated with a lower incidence of unscheduled bleeding/spotting days than the comparator. In addition, absence of withdrawal bleeding (also called amenorrhea) was much lower with the E4 containing preparations, particularly E4 associated with DRSP, than with the comparator. Finally, mean number of days with unscheduled bleeding/spotting by cycle was also lower with the combination of 15 mg E4/DRSP in comparison with the E2V/DNG preparation. This was also the case when compared to publicly available data on a marketed combined oral contraceptive containing E2 as estrogen in association with nomegestrol acetate (NOMAC).

Besides, the daily use of currently marketed estrogens (ethinylestradiol (EE), E2, E2V, conjugated equine estrogens) is associated with a dose-proportional increase in triglycerides levels. In the human body, high levels of triglycerides in the bloodstream have been linked to atherosclerosis and, by extension, the risk of heart disease and stroke. In the opposite to the currently available estrogens, E4 minimally increases triglycerides levels even at higher dosages.

In a study published after the priority date, Kluft et al. reported the observation of reduced haemostatic effects with COCs using estetrol as the estrogen by comparison with a COC using EE as the estrogen (Kluft et al., Reduced hemostatic effects with drospirenone-based oral contraceptives containing estetrol vs. ethinyl estradiol, Contraception, 95 (2017), p. 140-147) (published on-line Sep. 1, 2016). In this publication, both the estetrol-based COC and the comparator (YAZ® commercial product) used drospirenone as the progestogen. A conclusion that can be drawn from this study is that the estrogen estetrol offers a safer haemostatic profile than the estrogen ethinyl estradiol, when both are associated with drospirenone.

Regarding the effect of the progestogen on the safety profile, as discussed above and reported by Kluft et al. (page 141, left-hand column, beginning of 4^{th} paragraph), “markers and variables are notably less modified with LNG compared to [ . . . ] DRSP.” For this reason, and even though the Kluft at al. publication reports a better safety profile when EE is replaced by estetrol in COCs using DRSP as the progestogen, it was unexpected before the present invention that a COC using estetrol and DRSP would compare favourably to a 2^{nd} generation COC, such as one using LNG as the progestogen. In particular, the Kluft at al. publication does not disclose nor suggest that a composition comprising an estetrol component and DRSP as the progestogenic component reduces the blood clots risk to a level which is lower than the lower limit for blood clots risk associated with DRSP-based COCs. In view of the common general knowledge that a fourth generation COC (DRSP-based), despite having a number of advantages in terms of reduced side-effects, presents a much higher blood clots risk than an earlier generation COC, it was not conceivable before the present invention that a DRSP-based COC could present a blood clots risk outside the range of the risk associated with DRSP-based COCs of the prior art.

Priority applications WO2018/024912 and WO2018/065076 disclose the use of estetrol to alleviate the symptoms of dysmenorrhea. These applications are silent about the reduction of blood clot risk. Table 7 in these applications present the same clinical data as Table 1 of the Kluft at al. publication, although in a slightly different manner: while the Kluft at al. publication presents median values (expressed as percentage of the baseline value, taken as 100%), the patent applications present mean values (percentage changes).

As illustrated in Example 4 of priority application U.S. Ser. No. 16/323,110 (corresponding to WO 2018/024912), from the clinical results obtained with combinations of E4 and DRSP, the changes in the surrogate markers of VTE were minimal in comparison to the changes observed with Yaz® (a combination of 20 μg EE and 3 mg DRSP). DRSP is a fourth generation progestin associated with the highest risk of VTE when it is combined with the synthetic estrogen EE. Accordingly, the changes in the surrogate markers of VTE seen with a combination of EE and DRSP are substantial. In comparison, the changes observed with the E4 combinations are minimal even when DRSP is associated to the estrogen. For example, the SHBG plasma level changes observed when E4 was associated with 3 mg DRSP were considerably lower (mean percentage change of 7.9% for the 5 mg E4/3 mg DRSP group and of 44.5% for the 10 mg E4/3 mg DRSP group at treatment cycle 3) than the SHBG increases observed with a combination of 20 μg EE and 3 mg DRSP (mean percentage change of 306.3% for Yaz® at treatment cycle 3). The same positive pattern of change was observed with the 14 additional surrogate markers of VTE measured in this trial.

As discussed in priority application U.S. Ser. No. 16/323,110, in a comparative study it was surprisingly found that a combination of estetrol with drospirenone as the progestogenic component was more efficient at managing the symptoms of dysmenorrhea than a combination of estetrol with levonorgestrel as the progestogenic component. This is illustrated by the results of the clinical trial reported in Example 2 of priority application U.S. Ser. No. 16/323,110 (corresponding to WO 2018/024912).

As illustrated in Example 5 below, the changes in the surrogate markers of VTE were minimal in comparison to the changes observed with Yaz® (a combination of 20 μg EE and 3 mg DRSP). DRSP is a fourth generation progestin associated with the highest risk of VTE when it is combined with the synthetic estrogen EE. Accordingly, the changes in the surrogate markers of VTE seen with a combination of EE and DRSP are substantial. In comparison, the changes observed with the estetrol component combination are minimal even though DRSP is associated to the estetrol component.

Additionally, it can be seen in Example 5 below that the estetrol and DRSP combination described herein compares favourably to the 2^{nd} generation COC comparator, consisting of 30 mcg of EE with 150 mcg of LNG (Melleva®). In view of the previously recognized doubling in VTE risk when LNG is substituted by DRSP, it is highly surprising to find such a result.

In particular, parameters such as APC resistance (for example ETP-based APC resistance) is mostly one order of magnitude lower (mean percentage change in Tables 2 and 3 below and in Table 5) with the estetrol-based regimen by comparison with ethinyl estradiol-based regimens.

Acquired activated protein C resistance (by opposition with mutation-related) has been shown to be an independent risk factor of venous thrombosis which increases in the population of COC users (Rosing et al., 1997, Br. J. Haematol. 97, 233-8). APC resistance determined with an ETP-based assay (like in the present application) is an excellent marker for the thrombogenicity of COC.

In addition, as can be seen in Tables 1 and 4 below, statistical significance (for the change from Baseline to Cycle 6) was found between the COC of the invention and both 2^{nd} generation (Melleva®) and 4^{th} generation (Yaz®) COCs for the following parameters: Plasminogen (%), t-PA (ng/mL), ETP-based APC-r, Prothrombin Fragment 1+2 (nmol/L), and Soluble E-Selectin (ng/mL).

Statistical significance (for the change from Baseline to Cycle 6) was found between the COC of the invention and 4^{th} generation (Yaz®) COC for the following parameters: Factor VII activity (%), Protein S (%), Protein S. free (%), Protein C activity (%), aPTT-APC (sec) and SHBG (nmol/L).

Additionally, the methods described herein were found to suppress ovulation in 100% of patients.

### Methods of Treatment

The present methods for contraception or the treatment of dysmenorrhea usually employ uninterrupted oral administration of the estetrol component and the progestogenic component during a period of at least 10 days, including at least 20 days.

In accordance with the methods disclosed herein, the estetrol and progestogenic components may be administered in separate dosage units. However, it is also possible and indeed very convenient to combine these two components into a single dosage unit.

The term “uninterrupted” as used in here, means that the components are administered at relatively regular intervals, with no (therapeutically) significant interruptions. Naturally, minor interruptions may occur that do not affect the overall effectiveness of the present method, and indeed such aberrations are encompassed by the present invention. In a specific embodiment, and describing the concept more arithmetically, the administration regimen is deemed to be continuous if the longest interval between 2 subsequent administrations is not more than 3.5 times as long as the average interval. For example said longest interval may be not more than 2.5 times, or not more than 1.5 times, as long as the average interval.

In the methods disclosed herein, the combination of the progestogenic and estetrol component is suitably administered uninterruptedly during a period of at least 10 days.

The methods disclosed herein may suitably be reduced to practice in the form of a variety of administration methods (regimens). Amongst these methods are the so called “combined” methods. The combined methods make use of monophasic preparations, which contain dosage units with a constant amount of an estrogen and a progestogen, or bi- or triphasic preparations which have varying levels of estrogen and progestogen; in most cases consisting of relatively constant levels of estrogen with a step-wise increase in progestogen throughout the cycle. The combined methods have in common that they are based on a regimen which involves an administration-free interval of about 7 days whereby withdrawal bleeding, simulating the natural menses, occurs. Thus 21 day intervals of hormone administration alternate with 7 days during which no hormones are administered.

In specific embodiments, an administration-free interval of about 4 days is used. In these embodiments, a 24 day interval of hormone administration alternates with 4 days during which no hormones are administered.

In yet other specific embodiments, a 24 day interval of hormone administration during which an estetrol component and a progestogenic component are administered alternates with 4 days during which only an estetrol component is administered (from day 25 to day 28).

As an alternative to the aforementioned combined methods, the so called “sequential” administration method has been proposed, and can be implemented for the methods disclosed herein. Typical of the sequential method is that it comprises two consecutive phases, i.e. one phase during which estrogen and no progestogen is administered and another phase during which a combination of estrogen and progestogen is administered. The first sequential methods, like the aforementioned combined methods, made use of an administration free interval of about 7 days. More recently, sequential methods have been proposed which do not include an administration-free (or placebo) period, meaning that estrogen is administered throughout the full cycle and that progestogen is co-administered during only part of that cycle. WO 95/17895 (Ehrlich et al.) describes such an uninterrupted sequential method.

Yet another example of an administration method which is encompassed by the present disclosure is the so called “continuous combined” method, which is a particular version of the combined method that uses uninterrupted combined administration of a progestogenic and an estrogenic component during a prolonged period of time, e.g. more than 50 days. In contrast to ordinary combined and sequential methods, no regular menses occur in the continuous combined method as the continuous administration of progestogen in the indicated amounts induces amenorrhoca.

In one embodiment of the methods disclosed herein, which relates to the continuous combined method, a method may comprise the uninterrupted oral administration of the combination of the estetrol component and the progestogenic component during a period of at least 28, including at least 60 days.

In one specific embodiment of the continuous combined method, one tablet comprising the combination of the estetrol component and of the progestogenic component is initially taken daily for at least 24 consecutive days. Subsequently, during days 25 to 120, the patient may decide to take a 4-day tablet-free break. Said tablet-free break may not be longer than 4 days.

In any case, a 4-day tablet-free break has to be taken after 120 days of continuous tablet administration. After each 4-day tablet-free break, a new cycle starts with a minimum of 24 days and a maximum of 120 days of continuous administration.

In another embodiment, which relates to sequential and combined administration methods that employ a significant administration-free interval, a method as described herein may comprise an interval of at least 2 days, including from 3-9 days, including from 5-8 days, during which no progestogenic component and no estetrol component is administered and wherein the resulting decrease in serum concentration of the progestogenic component and the estetrol component induces menses.

Yet another embodiment, which concerns a sequential administration method without a significant pause, is characterised in that it comprises the uninterrupted oral administration of the estetrol component during a period of at least 28 days, including at least 60 days, and in that, following the combined administration of the estetrol component and the progestogenic component, the estetrol component and no progestogenic component are administered during 3-18 consecutive days, including during 5-16 consecutive days, and the resulting decrease in serum concentration of the progestogenic component should normally be sufficient to induce menses.

The contraceptive methods disclosed herein are capable of reducing the number, frequency and/or severity of adverse side effects including VTE, and ATE.

The methods of treating dysmenorrhea as disclosed herein, e.g., the methods for alleviating symptoms of dysmenorrhea, are capable of reducing the number, frequency and/or severity of adverse side effects including one or more of VTE, headache, breast pain, and the like. The methods are particularly useful for effective treatment of the symptoms of dysmenorrhea while reducing the side effect of VTE at a significantly low frequency and severity.

In particular embodiments, the methods described herein do not cause haemostatic change that exceeds the boundaries of the normal range. As used herein, “haemostatic change” is defined as the variation, upon administration of the compositions according to the invention, of the plasma level of one or more markers selected from: Sex Hormone Binding Globulin (SHBG), free tissue factor pathway inhibitor (free TFPI), free and total protein-S, protein-S activity, Corticosteroid Binding Globulin (CBG), Ceruloplasmin, antithrombin III, activated protein C (APC) resistance (e.g. APTT-based APCr or ETP-based APCr), Protein-C activity, D-dimer, Prothrombin, Prothrombin activity, Prothrombin fragment 1+2, Factor VII, Factor VIII, von Willebrand factor, Factor II, PAI-1, tissue-type plasminogen (t-PA), plasminogen, E-selectin, and fibrinogen. The above-listed markers and methods for the determination of their level are known in the art.

As used herein, the “normal range”, when referring to levels of haemostatic markers, refers to the prediction interval that 95% of the population fall into.

In some embodiments, the methods described herein do not cause haemostatic change exceeding the boundaries of the normal range after one cycle of treatment, after two cycles of treatment, or after three cycles of treatment.

In other particular embodiments, the methods do not cause a change in the level of protein-S which exceeds the boundaries of the normal range.

In other particular embodiments, the methods do not cause a change in the level of free TFPI which exceeds the boundaries of the normal range.

In specific contraceptive embodiments, a contraceptive method as described herein provides a better safety profile than currently available COCs, and in particular is associated with a lower risk of thromboembolic events, and may be employed in at-risk populations, such as first-ever users; switchers/re-starters with a break of >4 weeks (including a break of at least three months), and women with increased baseline risk for VTE due to one or more major risk factors (such as, but not limited to, BMI>30, older age, positive personal and/or family history, and risk factors such as those identified in Anderson and Spencer, 2003, Circulation, 107:1-9-1-16, “Risk factors for venous thromboembolism” which include, by way of example and not limitation, major surgery, prolonged immobility, childbirth, postpartum).

In relation to the at-risk population of older age, it is important to note that, as reported in Lidegaard et al. (2011) BMJ 343: d6423 (“Risk of venous thromboembolism from use of oral contraceptives containing different progestogens and oestrogen doses: Danish cohort study, 2001-9”), in particular in their Table 1 on page 9, VTE risk in non-users increases from 0.7 per 10,000 women year in the 15-19 years population to 5.8 per 10,000 women year in the 45-49 years population. For this latter population, COC use increases VTE risk by a factor of 3.6 (Table 1 of Lidegaard et al. 2011). The advantageous use of a COC as described herein is beneficial across all age groups, but significantly more so for the older age groups (e.g., age over 35) which have the highest incidence in absence of COC use, and whose incidence is additionally the most affected by use of a COC of the prior art.

### Compositions

In specific embodiments, a pharmaceutical composition for use in the methods described herein is formulated for daily administration, i.e. it represents a daily dosage unit.

In the case of oral administration, an oral dosage unit for use in the methods described herein may be a solid or semi-solid dosage form such as tablets, capsules, cachets, pellets, pills, powders and granules. The term “solid or semi-solid dosage form” also encompasses capsules that contain a liquid, e.g. an oil, in which the estetrol component is dissolved or dispersed.

Tablets and equivalent solid and semi-solid dosage forms can suitably contain materials such as binders (e.g. hydroxypropylmethyl cellulose, polyvinyl pyrrolidone, other cellulosic materials and starch), diluents (e.g. lactose and other sugars, starch, dicalcium phosphate and cellulosic materials), disintegrating agents (e.g. starch polymers and cellulosic materials) and lubricating agents (e.g., stearates and talc). These tablets and equivalent solid and semi-solid dosage forms may be prepared by wet granulation, e.g. using an aqueous solution or an organic solution, as well as by direct compression.

The estetrol component may be one or more substances selected from the group consisting of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms; and combinations thereof. In some embodiments, the estetrol component is estetrol (including estetrol hydrates). In some embodiments, the estetrol component contained in the dosage unit is estetrol monohydrate.

The estetrol component may be used at a daily dose of from 0.1 mg to 100 mg. The estetrol component may be used at a daily dose of from 1 mg to 40 mg. The estetrol component may be used at a daily dose of from 5 mg to 25 mg. The estetrol component may be used at a daily dose of from 10 mg to 20 mg.

In specific embodiments, the estetrol component is used at a daily dose of about 15 mg.

In other embodiments, dosages may be varied throughout the cycle (bi-phasic, tri-phasic or quadriphasic administration).

Examples of progestogenic components which may suitably be used in accordance with the methods described herein include: levonorgestrel, norgestimate, norethisterone, dydrogesterone, drospirenone, 3-beta-hydroxydesogestrel, 3-ketodesogestrel, 17-deacetylnorgestimate, 19-norprogesterone, acetoxypregnenolone, allylestrenol, amgestone, chlormadinone, cyproterone, demegestone, desogestrel, dienogest, dihydrogesterone, dimethisterone, ethisterone, ethynodiol diacetate, fluorogestone acetate, gastrinone, gestodene, gestrinone, hydroxymethylprogesterone, hydroxyprogesterone, lynestrenol, mecirogestone, medroxyprogesterone, megestrol, mele, gestrol, nomegestrol, norethindrone, norethynodrel, norgestrel (including d-norgestrel, and dl-norgestrel), norgestrienone, normethisterone, progesterone, quingestanol, (17 alpha)-17-hydroxy-11-methylene-19-norpregna-4, 15-dien-20-yn-3-one, tibolone, trimegestone, algestone-acetophenide, nestorone, promegestone, 17-hydroxyprogesterone esters, 19-nor-17hydroxyprogesterone, 17alpha-cthynyltestosterone, 17alpha-ethynil-19-nortestosterone, d-17beta-acetoxy-13beta-ethyl-17alpha-ethynylgon-4-en-3-one oxime, 6beta, 7beta; 15beta, 16beta-dimethylene-3-oxo-17-pregna-4,9(11)-diene-21, 17beta-carbolactone or tanaproget, and precursors of these compounds that are capable of liberating these progestogens in vivo when used in the methods described herein.

In specific embodiments, the progestogenic component is selected from the group consisting of progesterone, desogestrel, gestodene, dienogest, levonorgestrel, norgestimate, norethisterone, drospirenone, trimegestone, dydrogesterone, precursors of these progestogens, and mixtures of any two or more thereof.

When the progestogenic component is drospirenone, it may be used at a daily dose of from 0.5 mg to 10 mg, or from 1 mg to 4 mg. In specific embodiments, the progestogenic component of the invention is drospirenone and is used at a daily dose of about 3 mg.

When a different progestogenic component is used, the daily dose is adjusted such as to give the same pharmacological effect as a dose of 0.5 mg to 10 mg of drospirenone, or to give the same pharmacological effect as a dose of 1 mg to 4 mg of drospirenone.

In specific embodiments, estetrol is used at a daily dose of from 5 mg to 25 mg with drospirenone at a daily dose of 0.5 mg to 10 mg. In more specific embodiments, estetrol is used at a daily dose of from 10 mg to 20 mg with drospirenone at a daily dose of 1 mg to 4 mg. In further specific embodiments, estetrol is used at a daily dose of about 15 mg with drospirenone at a daily dose of about 3 mg. In further specific embodiments, the estetrol is used at a daily dose of about 5 mg with drospirenone at a daily dose of about 3 mg. In further specific embodiments, estetrol is used at a daily dose of about 10 mg with drospirenone at a daily dose of about 3 mg. In any of these embodiments, the estetrol and drospirenone may be formulated and administered in separate compositions, or in the same composition.

The methods and compositions have been described above with reference to a number of exemplary embodiments. Modifications and alternative implementations of some parts or elements are possible, and are included in the scope of the invention.

### Example 1

In this clinical study comparing the combination of estrogenic component and progestogenic component according to the invention with a commercially available contraceptive treatment using also a natural estrogen (estradiol valerate at a 1, 2 or 3 mg dose of with dienogest at a dose of 0, 2 or 3 mg, marketed as QLAIRA® by Bayer HealthCare, Germany), the number of drug-related adverse events (Treatment-emergent adverse events (TE-AE) reported by at least 2 subjects in any treatment group) and the levels of the SHBG marker were monitored.

As illustrated in the table below, 13 subjects (corresponding to 16.7%) reported TE-AEs related to headache in the treatment arm with the commercial product based on estradiol valerate and dienogest, while only 6 subjects (corresponding to 7.6%) did so in the group treated with a combination of 15 mg of estetrol and 3 mg of drospirenone. The number of events related to headache was thus shown to be much lower for the treatment according to the invention. In addition, the number of adverse events related to breast pain was similar and very low (only 1.3% of occurrences) for the two treatments.

Number (%) of subjects

| | Qlaira | 15 mg E4/ |
|---|---|---|
| Drug-related adverse | E2V/DNG | 3 mg DRSP |
| event | N = 78 | N = 79 |
| Headache | 13 (16.7) | 6 (7.6) |
| Breast pain | 1 (1.3) | 1 (1.3) |

In addition, in this study, changes in SHBG concentrations were sequentially assessed at baseline and during Cycle 4 and Cycle 6 of administration of the combinations of E4/DRSP and E2V/DNG to women starting combined contraception (groups of patient called “Starters”). Women were defined as Starters when they had not used a hormonal contraceptive in the 3 months prior to randomisation. This “wash-out” period allowed to exclude patients whose SHBG levels were influenced by the previous COC used. The results in terms of changes from baseline are shown below.

Mean change from Baseline (%)

| | Qlaira | 15 mg E4/ |
|---|---|---|
| SHBG level | E2V/DNG | 3 mg DRSP |
| (Starters) | N = 22 | N = 30 |
| Cycle 4/Screening | +43.9% (+/−35.6) | +27.1% (+/−12.1) |
| Cycle 6/Screening | +54.4% (+/−15.9) | +41.7% (+/−36.5) |

As is apparent from these results, the method of the invention permits to minimize the SHBG level changes from baseline both at Cycle 4 and at Cycle 6, compared to a commercially available COC which also uses a natural estrogen.

### Example 2

In this clinical study comparing two combination of estrogenic component and progestogenic component according to the invention with a commercially available contraceptive treatment (using ethinyl estradiol at 20 μg with drospirenone at 3 mg, marketed as YAZ® by Bayer HealthCare, Germany), several haemostasis markers as well as carrier proteins were measured and changes from baseline to end of Cycle 3 in these parameters are presented below.

Mean (SD) percentage change from baseline to end of treatment Cycle 3 for haemostasis parameters and carrier proteins, in women using a combination of E4/DRSP or EE/DRSP

| | Mean (SD) percentage change | | |
|---|---|---|---|
| | 5 mg E4/DRSP | 10 mg E4/DRSP | 20 μg EE/DRSP |
| Parameters | (n = 17) | (n = 19) | (n = 20) |

Molecular markers

| | | | | | | |
|---|---|---|---|---|---|---|
| D dimer | −25.9 | (32.71) | −22.0 | (29.70) | 35.8 | (56.14) |
| Prothrombin | −24.1 | (15.97) | −1.3 | (28.63) | 63.4 | (50.21) |
| fragment 1 + 2 | | | | | | |

Group 1 coagulation inhibition

| | | | | | | |
|---|---|---|---|---|---|---|
| Antithrombin III | 1.6 | (8.23) | 1.5 | (11.36) | −5.2 | (7.88) |
| APC resistance | 3.0 | (26.19) | 6.6 | (37.45) | 227.5 | (181.27) |
| (Rosing) | | | | | | |
| Protein S activity | 8.6 | (11.78) | 5.2 | (10.29) | −27.4 | (10.88) |
| Free TFPI | −13.5 | (15.33) | −15.1 | (8.89) | −46.6 | (8.50) |

Group 2 coagulation inhibition

| | | | | | | |
|---|---|---|---|---|---|---|
| Protein C activity | −3.7 | (9.95) | −0.5 | (10.18) | 15.8 | (13.82) |
| APC sensitivity | 0 | (9.77) | −2.7 | (8.88) | −9.8 | (9.74) |
| (APTT) | | | | | | |

Liver factors

| | | | | | | |
|---|---|---|---|---|---|---|
| Fibrinogen | 5.8 | (12.24) | −0.6 | (15.37) | 19.5 | (23.78) |
| Prothrombin | 11.7 | (34.71) | 21.4 | (24.98) | 13.8 | (21.76) |

Endothelial factors

| | | | | | | |
|---|---|---|---|---|---|---|
| Eselectin | 3.2 | (10.45) | −6.7 | (14.00) | −19.5 | (8.06) |
| tPA | −8.4 | (25.76) | −15.7 | (24.25) | −45.8 | (14.05) |

Carrier proteins

| | | | | | | |
|---|---|---|---|---|---|---|
| CBG | 17.1 | (16.64) | 28.1 | (19.55) | 170.3 | (75.60) |
| SHBG | 7.9 | (26.25) | 44.5 | (34.12) | 306.3 | (117.70) |
| Ceruloplasmin | 8.2 | (12.24) | 16.1 | (11.14) | 69.0 | (22.93) |

E4, estetrol;
EE, ethinylestradiol;
DRSP, drospirenone;
APC, activated protein C;
TFPI, tissue factor pathway inhibitor;
tPA, tissue type plasminogen;
CBG, corticosteroid binding globulin;
SHBG, sex hormone binding globulin.

A large difference was observed between DRSP combinations containing 20 μg EE and those with 5 or 10 mg E4: the procoagulant marker Prothrombin Fragment 1+2 plasma levels were decreased with the different E4/DRSP combinations, whereas they increased with EE/DRSP (+63% from baseline to 3 months of use). These opposite results indicate that increase in the thrombosis marker Prothrombin Fragment 1+2 is bound to the type (and dose) of estrogens (here EE vs. E4). Moreover, natural anticoagulants were unchanged (antithrombin III, protein S activity) or slightly decreased (free TFPI) by combinations containing E4 and typically decreased by EE/DRSP. While, as usual, activated partial thromboplastin time (APTT) related sensitivity to APC was almost unchanged with all preparations, the normalized APC sensitivity ratio was unchanged with E4 combinations whereas resistance to protein C was strongly increased by the EE/DRSP combination. Simultaneously to the non-increase of the procoagulant markers when using combinations of E4 and DRSP, there was a slight decrease in fibrinolysis parameters such as tPA and D-dimers levels.

With EE/DRSP combination, the increase in SHBG was important (+306%). All combinations of E4 (5, 10 mg) with DRSP showed a moderate increase in SHBG. Note that SHBG is considered as the most relevant biomarker for estrogenic impact of a COC on liver metabolism (Odlind V. et al.; Acta Obstet Gynecol Scand 2002; 81:482).

CBG and ceruloplasmin are essentially synthesized under the influence of estrogens and are much less sensitive to the androgenic action of progestins. In the E4 and DRSP groups, increasing the dose of estrogen resulted in a slight increase from baseline for CBG and ceruloplasmin. However, by far the largest change from baseline was observed in the EE/DRSP group compared to the E4 treatment groups.

### Example 3

A multicenter, placebo-controlled, randomised study to evaluate the benefits of the method of the invention on alleviating complaints of dysmenorrhea was conducted. The study population consisted in healthy female subjects, between 12 and 35 years old, inclusive (at the time of screening), with primary dysmenorrhea (onset <3 years post menarche).

The product according to the method of the invention was a combination tablet with estetrol (15 mg) and drospirenone (3 mg) administered orally once daily in continuous or 24/4-day regimen (i.e. 24 days of active tablets followed by 4 days of placebo tablets). Other doses of estetrol were included in supplementary arms, in addition to the placebo arm.

Efficacy was demonstrated by following the change between baseline evaluation period and treatment evaluation period, primarily in the number of days with dysmenorrhea pain. Dysmenorrhea pain was defined as pelvic pain during the menstrual/withdrawal bleeding episode and the 2 days before this episode. Secondarily, the efficacy was followed by a daily scoring of dysmenorrhea pain, according to the following scale:
0—No pain;
1—Mild pain with no need for painkiller;
2—Moderate pain with need for painkiller;
3—Severe pain with need for painkiller.

Additional efficacy assessment were made as follows:
1. Change Between Baseline Evaluation Period and End of Treatment Evaluation Period in Number of Days With Pelvic Pain Independent of Occurrence of Vaginal Bleeding;
2. Change Between Baseline Evaluation Period and Treatment Evaluation Period in Number of Days With Pelvic Pain During Unscheduled Bleeding;
3. Change Between Baseline Evaluation Period and Treatment Evaluation Period in Rescue Medication Use. Rescue medication use will be standardized intake of 200 mg Ibuprofen tablets;
4. Percentage of Participants With Interference of Dysmenorrhea Pain With Work/School and Social or Other Activity;
5. Percentage of Participants and Hours/Days of Missing Time From Work Due to Dysmenorrhea Pain at Baseline, Month 3 and Final Examination (after Month 6);
6. Percentage of Participants Satisfied With Study Treatment;
7. Own Costs of Physiotherapy, Alternative Medicine, Acupuncture, Osteopathy, Medical Counselling, Massages, Herbal supplements/Teas per treatment of dysmenorrhea pain evaluated by completion of a Resource Use Questionnaire (converted to euros);
8. Patient's improvement during the course of study as per The Clinical Global Impression Scale (CGI) completed by Investigators;
9. Participants' Assessment in the Clinical Global Impression The Clinical Global Impression Scale (CGI) as completed by the participants and rating their improvement during the course of the study;
10. General Health, Body Pain, Physical and Social Functioning, Mental Health and Vitality as measured by General Health and Well-being Questionnaire SF-36 at Baseline, Month 3 and at Final Examination, using the SF-36 self-administered questionnaire, a general health status measure used to evaluate patient populations and to compare health status across different populations.

The clinical study demonstrates that the product according to the invention is effective in improving the symptoms of dysmenorrhea.

### Example 4

A multi-institutional, placebo-controlled trial was conducted with collaborative randomized allocation double-blinded control for dysmenorrhea patients (primary dysmenorrhea patients, and secondary dysmenorrhea patients) aged 16 and older. The study drug is a combination tablet containing estetrol (15 mg) and drospirenone (3 mg).

The tablet has two modes of administration:
for cyclic administration, the study drug was given orally in a cycle consisting of administration of one tablet per day at the same time every day for 24 days, followed by a 4-day discontinuation period;
for continued administration, the study drug was continuously administered, without discontinuation, in a dose of one tablet per day at the same time every day.

For primary evaluation, changes from baseline were scored at week 16 (4 cycles) according to the evaluation scale below.

| | Grade | Score | Details |
|---|---|---|---|
| Dysmenorrhea | None | 0 | none |
| (or nonmenstrual | Mild | 1 | some loss of work (or study) |
| pelvic pain) | | | efficiency |
| | Moderate | 2 | want to take some rest in bed, |
| | | | loss of work |
| | Severe | 3 | in bed more than 1 day |
| Use of analgesics | None | 0 | none |
| (previous or | Mild | 1 | take analgesics for 1 day |
| present period) | Moderate | 2 | take analgesics for 2 days |
| | Severe | 3 | take analgesics for > 3 days |

Dysmenorrhea score involving severity of dysmenorrhea as well as use of analgesics as reported in Harada T et al., Low-dose oral contraceptive pill for dysmenorrhea associated with endometriosis: a placebo-controlled, double-blind, randomized trial, Fertil Steril 2008; 90:1583-1588

Additional efficacy assessments were made as follows:
1. dysmenorrhea pain was evaluated by observing changes from baseline using a VAS scale and pelvic pain scores;
2. occurrence of abnormal vaginal bleeding was evaluated;
3. amelioration of premenstrual syndrome was evaluated by observing changes from baseline using a self-administered questionnaire;
4. potential risk of the study drug on venous thromboembolism (VTE) was evaluated using surrogate markers of VTE (e.g., D-dimer, SHBG, protein C activity, and protein S activity);
5. changes in the severity of lower abdominal pain, lower back pain, headache, vomiting, and a feeling of sickness during menstruation;
6. changes in endometrial thickness from baseline;
7. serum CA125 concentration, and serum C reactive protein concentration;
8. serum estradiol concentration, and serum progesterone concentration;
9. safety items: adverse events; clinical test results (including an endocrine test), vital signs; uterine size.

The clinical study demonstrates that the product according to the invention is effective in improving the symptoms of dysmenorrhea.

### Example 5

### Study Protocol

A single center, randomized, open-label, controlled, three-arm study to evaluate the effect of a Combined Oral Contraceptive (COC) containing 15 mg estetrol (E4) and 3 mg drospirenone (DRSP) and of two reference COCs containing either 30 mcg ethinylestradiol (EE) and 150 mcg levonorgestrel (LNG) or 20 mcg EE and 3 mg DRSP on endocrine function, metabolic control and haemostasis during 6 treatment cycles was conducted.
Study Type: Interventional (Clinical Trial)
Actual Enrollment: 101 participants
Allocation: Randomized
Intervention Model: Parallel Assignment
Masking: None (Open Label)
Primary Purpose: Prevention

| Arm | Intervention/treatment |
|---|---|
| Experimental: | 15 mg E4 combined with 3 mg DRSP |
| 15 mg E4/3 mg | administered in a 24/4-day regimen (i.e. |
| DRSP combined | 24 days of pink active tablets followed |
| oral contraceptive | by 4 days of white placebo tablets). |
| | One tablet per day orally for 6 treatment cycles. |
| Active Comparator: | 30 mcg EE combined with 150 mcg LNG |
| 30 mcg EE/150 mcg | administered in a 21/7-day regimen (i.e. |
| LNG combined | 21 days of yellow active tablets followed |
| oral contraceptive | by 7 days of white placebo tablets). |
| | One tablet per day orally for 6 treatment cycles. |
| Active Comparator: | 20 mcg EE combined with 3 mg DRSP |
| 20 mcg EE/3 mg | administered in a 24/4-day regimen (i.e. |
| DRSP combined | 24 days of pink active tablets followed |
| oral contraceptive | by 4 days of white placebo tablets). |
| | One tablet per day orally for 6 treatment cycles. |

### Primary Outcome Measures:

For each of the parameters 1 to 16 listed below, the following applies:

[Time Frame: Between Days 18 and 21 for the pretreatment Cycle, and between Days 18 and 21 for the Cycles 3 and 6 (1 cycle=28 days).]
1. Plasma concentration of prothrombin fragment 1+2
2. Plasma concentration of APC resistance (ETP-based, APTT-based)
3. Plasma concentration of D-dimer
4. Plasma concentration of factor VII
5. Plasma concentration of factor VIII
6. Plasma concentration of von Willebrand factor
7. Plasma concentration of factor II
8. Plasma concentration of antithrombin
9. Plasma concentration of free and total Protein S
10. Plasma concentration of protein C
11. Plasma concentration of plasminogen activator inhibitor type-1 (PAI-1)
12. Plasma concentration of tissue type plasminogen activator (t-PA)
13. Plasma concentration of plasminogen
14. Plasma concentration of free tissue factor pathway inhibitor (TPFI)
15. Plasma concentration of E-selectin
16. Plasma concentration of fibrinogen

For each of the parameters 17 to 21 listed below, the following applies:

[Time Frame: At screening, between Days 18 and 21 for the pretreatment Cycle, and between Days 18 and 21 for Cycles 3 and 6 (1 cycle=28 days).]
17. Serum concentration of insulin
18. Serum concentration of glucose
19. Serum concentration of C-peptide
20. Plasma concentration of glycated hemoglobin (HbA1c)
21. Homeostasis Model Assessment-Insulin Resistance (HOMA-IR)
22. Oral glucose tolerance test (OGTT) [Time Frame: At 0 (pre-glucose challenge), 30, 60, 90, 120 and 180 minutes after glucose challenge during pretreatment Cycle; at 0 (pre-glucose challenge), 30, 60, 90, 120 and 180 minutes after glucose challenge during Cycles 3 and 6 (1 cycle=28 days).]

For each of the parameters 23 to 35 listed below, the following applies:

[Time Frame: Between Days 18 and 21 for the pretreatment Cycle, and between Days 18 and 21 for Cycles 3 and 6 (1 cycle=28 days).]
23. Serum concentration of prolactin
24. Serum concentration of follicle-stimulating hormone (FSH)
25. Serum concentration of luteinizing hormone (LH)
26. Serum concentration of estradiol (E2)
27. Serum concentration of progesterone (P)
28. Serum concentration of thyroid stimulating hormone (TSH)
29. Serum concentration of free thyroxine (fT3)/free triiodothyronine (fT4)
30. Serum concentration of dihydroepiandrostenedione (DHEAS)
31. Serum concentration of androstenedione
32. Serum concentration of total testosterone (T)
33. Serum concentration of dihydrotestosterone (DHT)
34. Serum concentration of total cortisol
35. Serum concentration of aldosterone

For each of the parameters 36 to 39 listed below, the following applies:

[Time Frame: At screening, between Days 18 and 21 for the pretreatment Cycle, and between Days 18 and 21 for the Cycles 3 and 6 (1 cycle=28 days).]
36. Serum concentration of high density lipoprotein (HDL)-cholesterol
37. Serum concentration of low density lipoprotein (LDL)-cholesterol
38. Serum concentration of total cholesterol
39. Serum concentration of triglycerides

For each of the parameters 40 to 47 listed below, the following applies:

[Time Frame: Between Days 18 and 21 for the pretreatment Cycle, and between Days 18 and 21 for the Cycles 3 and 6 (1 cycle=28 days).]
40. Serum concentration of lipoprotein (a)
41. Serum concentration of apoliporotein A1
42. Serum concentration of apoliporotein B
43. Serum concentration of C-reactive protein
44. Serum concentration of corticosteroid binding globulin (CBG)
45. Serum concentration of sex hormone binding globulin (SHBG)
46. Serum concentration of thyroxin binding globulin (TBG)
47. Serum concentration of angiotensinogen

### Secondary Outcome Measures:

1. Number of subjects with adverse events as a measure of safety and tolerability [Time Frame: From up to 28 days before randomization to maximum Day 4 of the Cycle 7 (1 cycle=28 days).]
2. Serum concentration of lactate dehydrogenase (LDH) 1 and 2 [Time Frame: Between Days 18 and 21 for the pretreatment Cycle and between Days 18 and 21 for the Cycle 6 (1 cycle=28 days)]
3. Serum concentration of tropinin T and I
   [Time Frame: Between Days 18 and 21 for the pretreatment Cycle and between Days 18 and 21 for the Cycle 6 (1 cycle=28 days)]
4. Electrocardiogram (ECG) parameters
   [Time Frame: At screening and between Days 18 and 21 for Cycle 6 (1 cycle=28 days).]

The following ECG parameters will be recorded: heart rate, PR-interval, QRS-duration, QT-interval, QTc interval (Fridericias's)
5. Echocardiographic parameters
   [Time Frame: At screening and between Days 18 and 21 for Cycle 6 (1 cycle=28 days).]
6. Change from baseline to end of treatment in the different items of the menstrual distress questionnaires (MDQ) form C
   [Time Frame: At pretreatment Cycle and between Days 18 and 21 for Cycle 6 (1 cycle=28 days).]

### Eligibility Criteria

Ages Eligible for Study: 18 Years to 50 Years (Adult)
Sexes Eligible for Study: Female
Accepts Healthy Volunteers: Yes

### Criteria

### Inclusion Criteria:

Healthy adult woman
Negative pregnancy test at subject screening and randomization
Aged 18-50 years (inclusive) at the time of signing the ICF
Good physical and mental health on the basis of medical, surgical and gynecological history, physical examination, gynecological examination, clinical laboratory, ECG, echocardiography and vital signs
BMI from 18.0 to 30.0 kg/m², inclusive, at time of screening visit
Able to fulfil the requirements of the protocol and have indicated a willingness to participate in the study by providing written informed consent

### Exclusion Criteria:

Known hypersensitivity to any of the investigational product ingredients
Smoking if >35 years old
Dyslipoproteinemia or use of antilipidemic agent
Known diabetes mellitus
Current use of antidiabetic drugs, including insulin
Arterial hypertension
Any condition associated with an increased risk of venous thromboembolism and/or arterial thromboembolism.
Any condition associated with abnormal uterine/vaginal bleeding.
Presence of an undiagnosed breast mass
Current symptomatic gallbladder disease
History of pregnancy- or COC-related cholestasis
Presence or history of severe hepatic disease
Presence or history of pancreatitis if associated with hypertriglyceridemia
Porphyria
Presence or history of benign liver tumors (focal nodular hyperplasia and hepatocellular adenoma)
Presence of renal impairment (glomerular filtration rate [GFR]<60 mL/min/1.73 m²)
Hyperkalemia or presence of conditions that predispose to hyperkalemia
Presence or history of hormone-related malignancy
History of non-hormone-related malignancy within 5 years before screening; subjects with a non-melanoma skin cancer are allowed in the study
Use of drugs potentially triggering interactions with COCs
History of alcohol or drug abuse within 12 months prior to screening
Presence or history of thyroid disorders
Participation in another investigational drug clinical study within 1 month (30 days) or have received an investigational drug within the last 3 months (90 days) prior to randomization. Subjects who participated in an oral contraceptive clinical study using Food and Drug Administration (FDA)/European Union (EU) approved active ingredients, may be randomized 2 months (60 days) after completing the preceding study
Sponsor, contract research organization (CRO) or Principal Investigator's (PI's) site personnel directly affiliated with this study
Is judged by the PI to be unsuitable for any reason

### Results

The following haemostasis parameters were determined at base line (no COC use) and after 3 cycles and 6 cycles of COC use:

Coagulation factors: fibrinogen, prothrombin, factor VII, factor VIII and von Willebrand factor (note: although classified in this group for ease of reference, the van Willebrand factor is actually not, per se, a coagulation factor).

Anticoagulant proteins: antithrombin, protein S (ELISA and activity), factor XIV=protein C, and TFPI.

Proteins involved in fibrinolysis: plasminogen, tissue plasminogen activator (tPA), plasminogen activator inhibitor-1 (PAI-1)

Functional clotting tests: aPTT-based activated protein C resistance (APCr), ETP-based activated protein C resistance, activated coagulation time-APC, activated coagulation time+APC.

Markers of ongoing coagulation: prothrombin fragment 1+2 and D-Dimer

Miscellaneous: soluble E-selectin and sex hormone binding globulin (SHBG).

With respect to the thrombogenicity of the COCs, the haemostasis parameters that are associated with an increased risk of venous thrombosis or that are so-called (surrogate) risk markers of venous thrombosis are:

Coagulation factors: fibrinogen, prothrombin, and factor VIII

Anticoagulant proteins: antithrombin, protein S (ELISA and activity), factor XIV=protein C, and TFPI.

Proteins involved in fibrinolysis: are in general not considered to be risk factors of VTE.

Functional clotting tests: aPTT-based activated protein C resistance (APCr), ETP-based activated protein C resistance.

Markers of ongoing coagulation: prothrombin fragment 1+2 and D-Dimer

Miscellaneous: sex hormone binding globulin (SHBG) is a surrogate marker of VTE risk in case of hormone use.

In the context of prothrombin, of particular interest is the publication by Poort et al. (A common genetic variation in the 3′-untranslated region of the prothrombin gene is associated with elevated plasma prothrombin levels and an increase in venous thrombosis, Blood, Vol. 88, N°10, 1996, pages 3698-3703). Table 2 on page 3701 of this publication establishes a relationship between the levels of prothrombin activity and the Odds Ratio for thrombosis risk. From this table, it can for example be seen that in case the prothrombin activity is increased by a level of between 5% to 15% above the normal (which is taken as 100%), the odds ratio of thrombosis risk is at 1.4. When the prothrombin activity level rises above 115%, the odds ratio changes to 2.1.

Because the actual population is in fact spread around the average value in a Gaussian distribution, apparently small changes in the position of the centre of the Gaussian (the average prothrombin level) imply significant changes in the number of subjects in the high risk category (the category appearing last in the Table 2 from Poort et al., i.e. the subjects displaying 115% and more as prothrombin level and thus having a 2.1 Odds ratio). For example, while in a group of subjects not taking COCs (and therefore displaying an average prothrombin level at 100%), the “tail” of the Gaussian curve corresponding to subjects at high risk (with an Odds Ratio of 2.1) represents 14% of the whole population, in a group of subjects taking a COC which displaces the average prothrombin level to 111% (as is the case in the data of the Example for the EE/LNG treatment, please refer to the “prothrombin activity” entry in Table 3 at cycle 6), the high risk patient population (with an Odds Ratio of 2.1) will very significantly increase to 38% of the whole population.

From this illustrative calculation, it can easily be understood that a small variation in the average level of a thrombotic parameter may in fact have a very large impact in terms of the number of subjects falling into the high risk category.

To summarise the effects the different COCs on the haemostasis parameters, for each parameter a number of different approaches were used. In a first method, the changes of the average measured levels amongst the subjects in a given group were calculated at cycle 3 and cycle 6 by comparison to the average levels at the baseline. The corresponding values are displayed in Table 1 below.

In a second approach, the changes at cycles 3 and 6 by comparison to the baseline were initially computed for each patient, and the averages of these changes was then calculated. The corresponding results are presented as percentages in Table 2 below.

Thirdly, the average measured levels at baseline and at cycles 3 and 6 were used to calculate the percentage of changes from baseline to cycles 3 and 6 which are presented in Table 3 below.

In the present study, the effects of EE/DRSP on haemostasis parameters are more pronounced than those of EE/LNG. Very surprisingly, however, the effects of E4/DRSP are comparable to those of EE/LNG (second generation COC). Actually, for most parameters the effect of E4/DRSP is even less than that of EE/LNG which suggests that E4/DRSP might be less thrombogenic than EE/LNG.

In relation to the Odds Ratio from the Poort et al. paper discussed above, it is important to note in Table 3 that the Prothrombin activity is found to increase by only 5.9% after 6 cycles when the method of the invention is used, whereas it is found to increase by 11.2% and 10.4% when EE/LNG and EE/DRSP are used, respectively. From this point of view, the method of the invention is shown to put a lot fewer subjects into the “at risk” category (defined as having an Odds Ratio of 2.1).

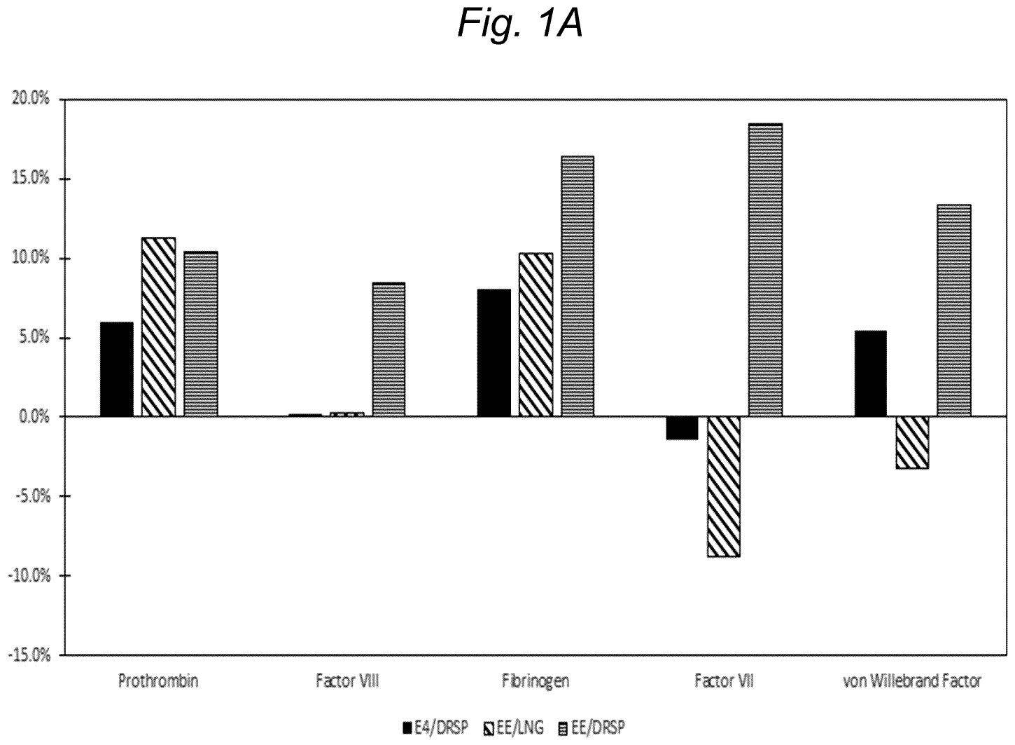

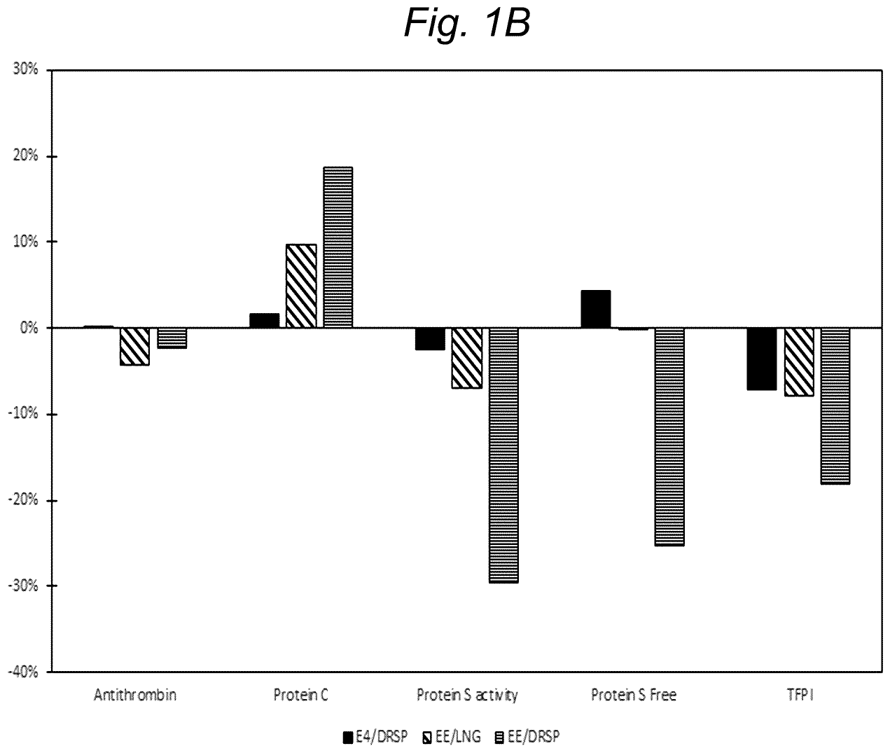

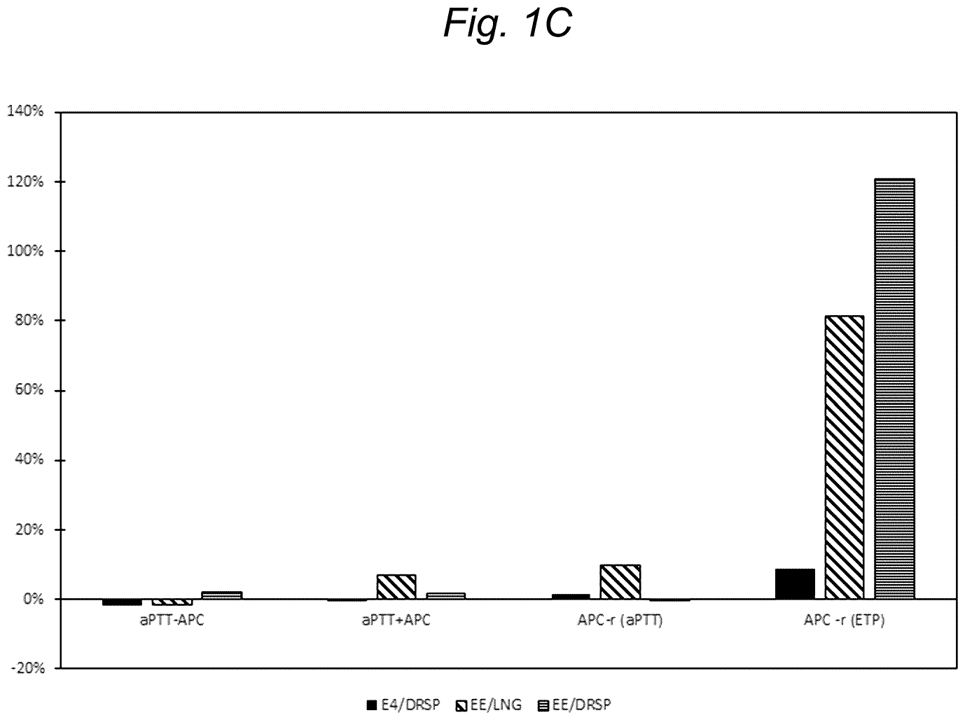

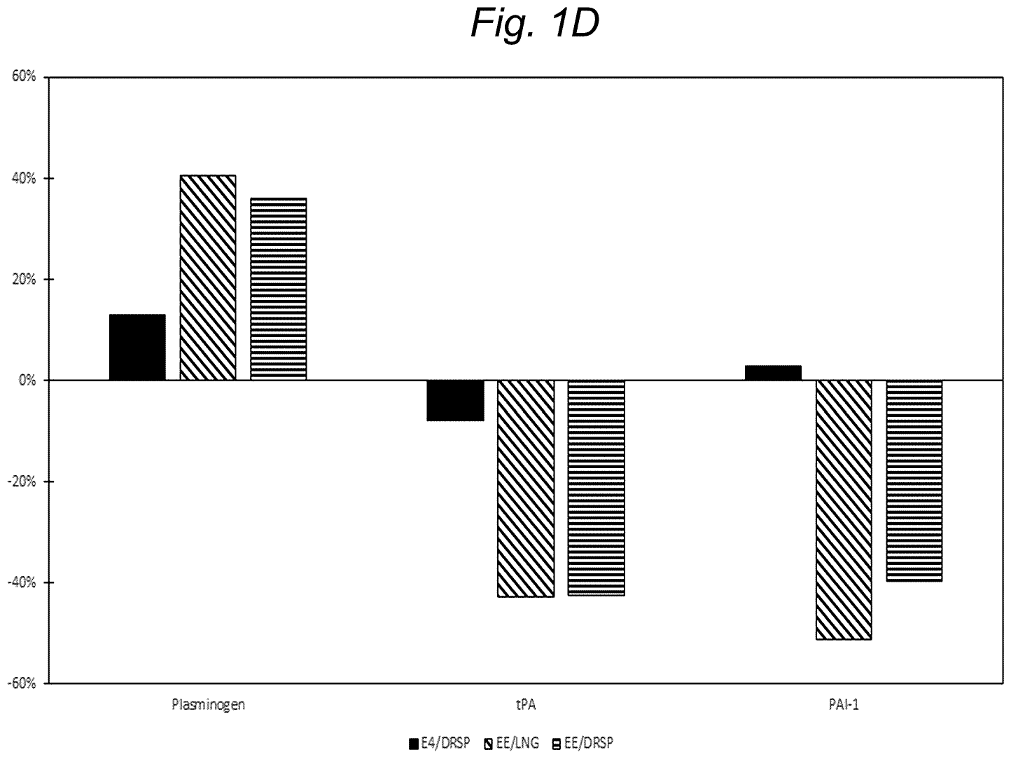

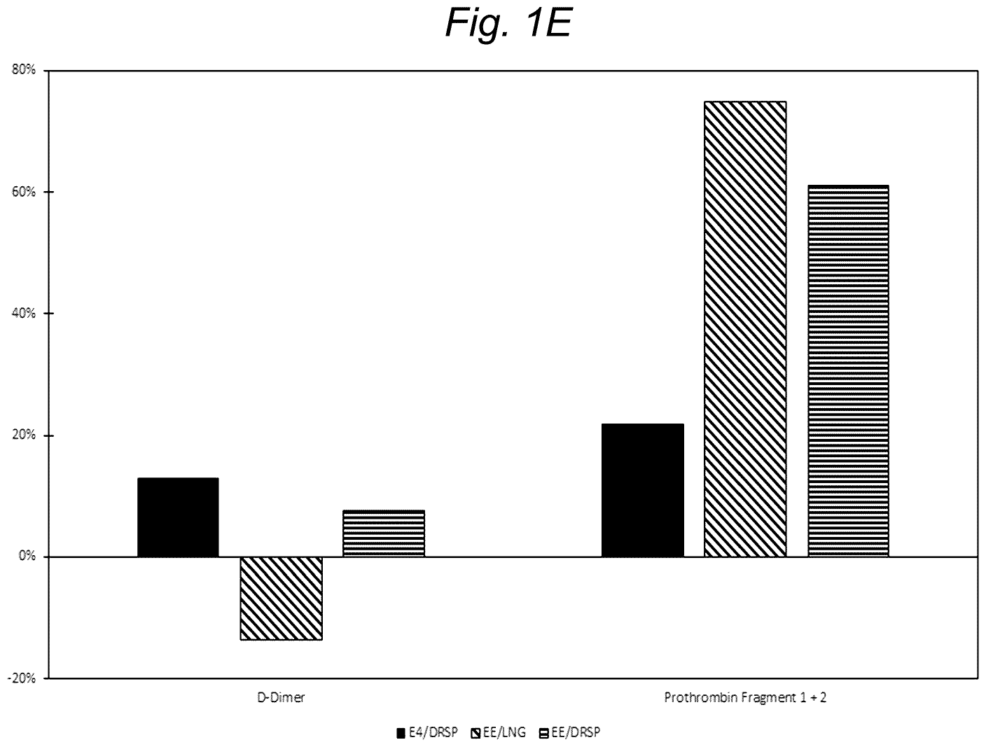

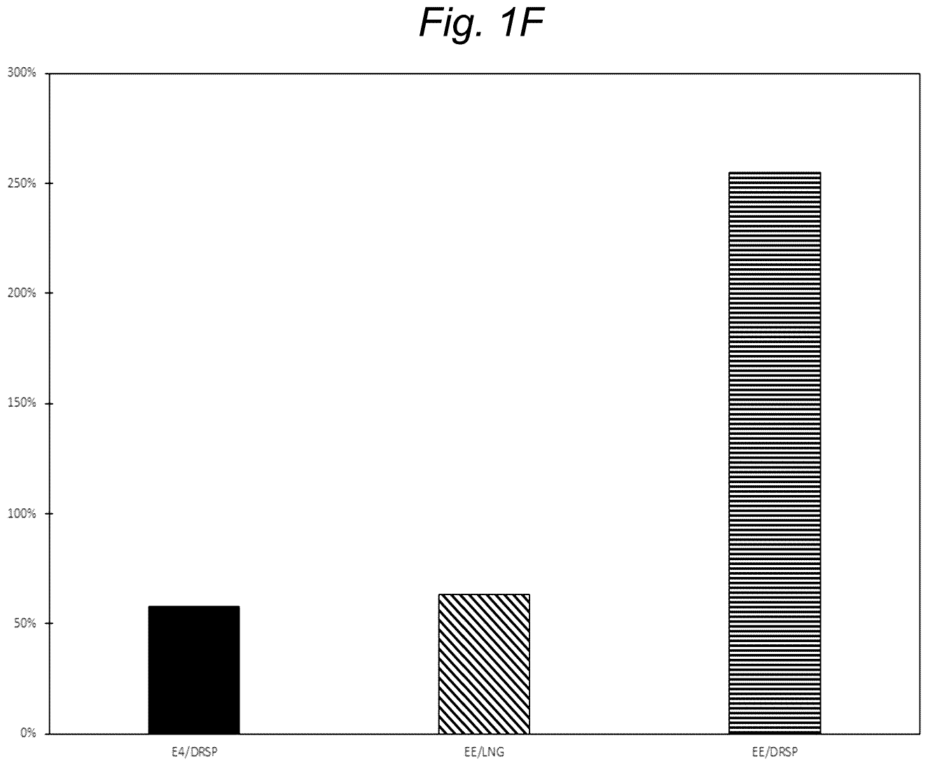

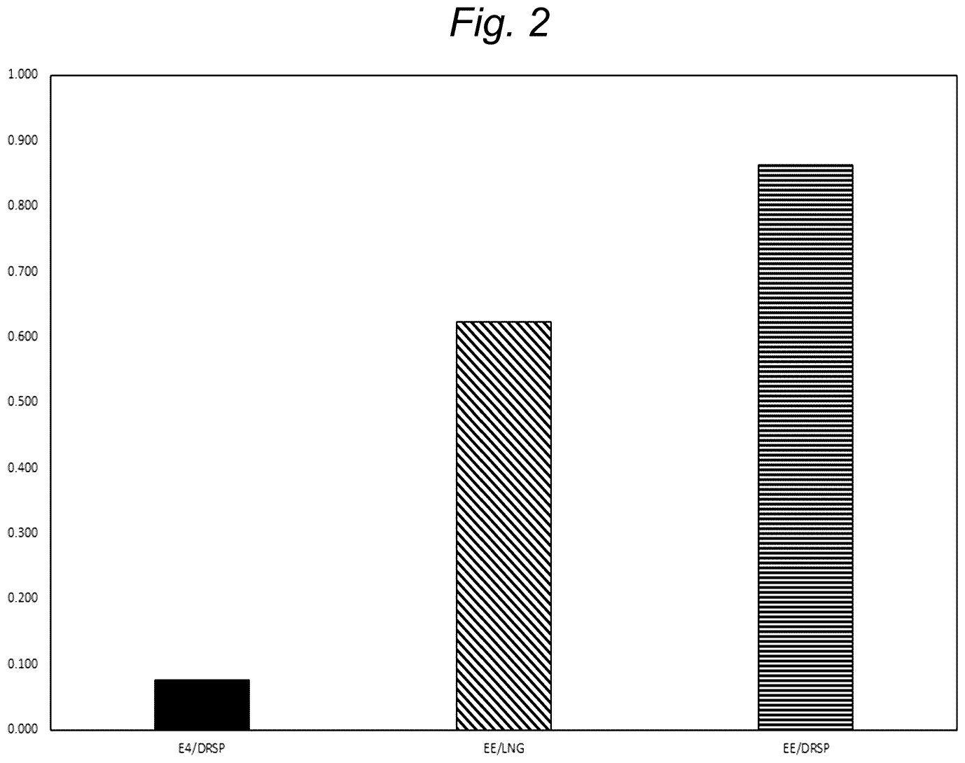

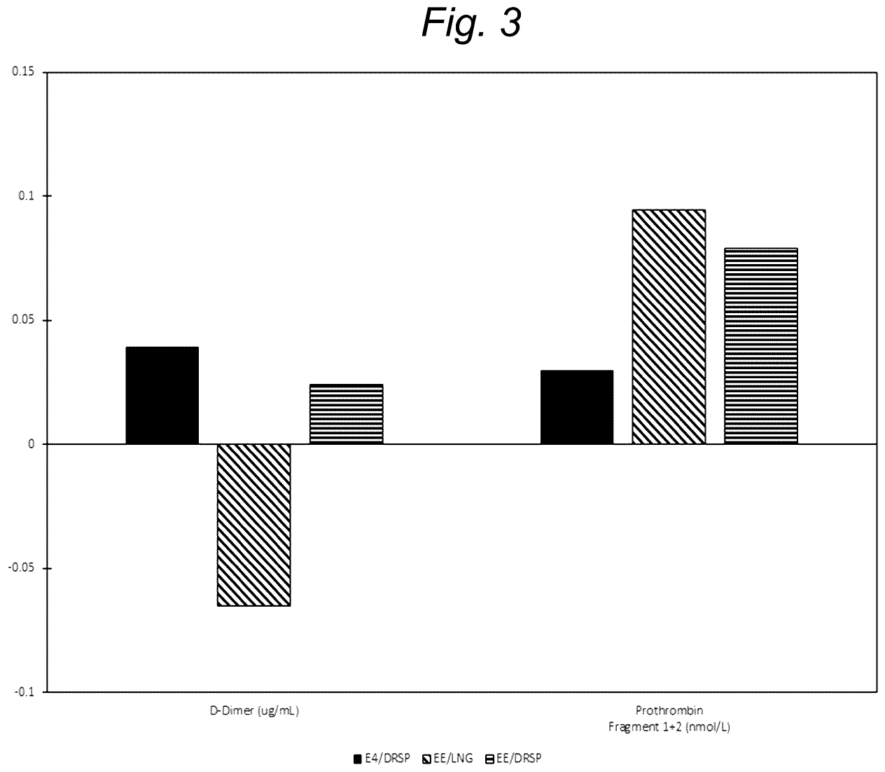

Similarly, it is known from Odlind et al. (Can changes in sex hormone binding globulin predict the risk of venous thromboembolism with combined oral contraceptive pills?, Acta Obstet Gynecol Scand, 2002; 81; pages 482-490) that the incidence of VTE is directly linked to the amount of increase in SHBG levels observed after COCs are administered. Based on the levels observed in Table 3, it can be seen that the method of the invention is estimated to induce a slightly lower risk than the EE/LNG COC, and induces a vastly lower risk than the EE/DRSP COC. In other words, the contraceptive method of the invention would be displayed in the FIG. 1 from Odlind et al. in the lower left corner of the graph, close to the “LNG” data point, and very remote from the “DSG/GST” and the “CPA” data points.

TABLE 1
Change from baseline (hereinafter, “BL”) to Cycles 3 and 6 in levels of various haemostasis parameters (per-protocol population)

| Table 1-Haemostasis parameters | E4/DRSP | | | EE/LNG | | | EE/DRSP | | |
|---|---|---|---|---|---|---|---|---|---|
| Mean (SD) | | Change BL | Change BL | | Change BL | Change BL | | Change BL | Change BL |
| Median (min-max) | Baseline | to Cycle 3 | to Cycle 6 | Baseline | to Cycle 3 | to Cycle 6 | Baseline | to Cycle 3 | to Cycle 6 |

Coagulation factors

| | | | | | | | | | | |
|---|---|---|---|---|---|---|---|---|---|---|
| Fibrinogen | mean change | 246.1 | 11.0 | 19.9 | 254.9 | 27.1 | 19.9 | 241.3 | 44.2 | 37.8 |
| (mg/dL) | (SD) | (42.24) | (48.27) | (40.62) | (77.32) | (71.74) | (73.45) | (47.63) | (60.06) | (59.86) |
| | median change | 240.0 | 9.0 | 27.0 | 239 | 22.0 | 12.0 | 228.5 | 57.5 | 36.5 |
| | (min-max) | (173-355) | (−77-162) | (−63-93) | (144-472) | (−179-205) | (−167-199) | (176-409) | (−107-159) | (−182-152) |
| Prothrombin activity | mean change | 85.7 | 5.2 | 5.1 | 89.0 | 11.7 | 9.5 | 90.0 | 12.9 | 9.4 |
| (%) | (SD) | (7.40) | (7.06) | (7.86) | (11.42) | (7.15) | (8.55) | (9.44) | (7.97) | (9.58) |
| | median change | 86.0 | 7.0 | 5.0 | 87.0 | 11.0 | 10.0 | 89.0 | 14.0 | 6.5 |
| | (min-max) | (67-106) | (−10-18) | (−10-20) | (72-112) | (−1-25) | (−8-31) | (77-112) | (1-27) | (−3-38) |
| Factor VII activity | mean change | 97.2 | −5.1 | −1.3 | 93.1 | −7.1 | −8.0 | 95.5 | 19.9 | 17.7 |
| (%) | (SD) | (22.80) | (11.32) | (15.78) | (18.26) | (14.89) | (14.07) | (17.59) | (19.27) | (15.84) |
| | median change | 94.0 | −4.0 | −3.0 | 93.0 | −7.0 | −4.0 | 93.5 | 18.0 | 17.5 |
| | (min-max) | (61-166) | (−33-22) | (−34-38) | (53-127) | (−46-23) | (−43-25) | (64-130) | (−12-68) | (−13-56) |
| | p-value* | | | | | | 0.3711 | | | 0.0001 |
| Factor VIII activity | mean change | 135.4 | 10.2 | 0.3 | 120.9 | 8.7 | 0.3 | 118.0 | 20.7 | 9.9 |
| (%) | (SD) | (47.62) | (28.16) | (28.91) | (34.88) | (29.47) | (35.79) | (33.49) | (25.10) | (30.56) |
| | median change | 114.0 | 11.0 | 6.0 | 115.0 | 9.0 | 3.0 | 110.0 | 19.0 | 7.0 |
| | (min-max) | (85-255) | (−62-68) | (−69-65) | (74-217) | (−77-72) | (−83-92) | (72-203) | (−19-56) | (−53-79) |
| vWF | mean change | 109.9 | 4.9 | 6.0 | 105.1 | −1.7 | −3.6 | 93.8 | 10.1 | 12.9 |
| (%) | (SD) | (38.31) | (17.14) | (20.66) | (46.84) | (18.94) | (20.15) | (24.48) | (14.32) | (19.10) |
| | median change | 103.0 | 4.0 | 6.0 | 96.0 | −2.0 | −2.0 | 88.0 | 8.0 | 10.0 |
| | (min-max) | (60-240) | (−50-38) | (−39-55) | (56-295) | (−60-30) | (−60-37) | (55-154) | (−13-40) | (−29-58) |
| | p-value* | | | | | | 0.1673 | | | 0.4016 |

Anticoagulant proteins

| | | | | | | | | | | |
|---|---|---|---|---|---|---|---|---|---|---|
| Antithrombin | mean change | 97.4 | −0.1 | 0.2 | 98.1 | −2.8 | −3.9 | 98.9 | −2.1 | −2.2 |
| (%) | (SD) | (6.75) | (5.41) | (5.09) | (11.71) | (6.33) | (6.43) | (6.66) | (7.06) | (8.43) |
| | median change | 96.0 | 0.0 | −1.0 | 99.0 | −2.0 | −5.0 | 96.5 | −2.5 | −3.5 |
| | (min-max) | (87-110) | (−11-12) | (−9-11) | (64-124) | (−21-12) | (−13-11) | (88-114) | (−13-13) | (−17-15) |
| Protein S | mean change | 94.5 | 0.6 | −2.3 | 104.1 | −1.2 | −7.4 | 104.9 | −25.0 | −31.8 |
| (%) | (SD) | (13.08) | (13.77) | (12.16) | (16.89) | (18.26) | (15.62) | (17.07) | (12.16) | (13.42) |
| | median change | 93.0 | 1.0 | −5.0 | 105.0 | −2.0 | −5.0 | 104.0 | −26.0 | −30.5 |
| | (min-max) | (74-134) | (−29-36) | (−22-26) | (82-142) | (−30-50) | (−41-29) | (65-149) | (−57-−6) | (−67-−8) |
| | p-value* | | | | | | 0.4564 | | | <0.0001 |
| Protein S. free | mean change | 85.2 | 6.5 | 3.7 | 95.6 | 2.3 | −1.1 | 91.0 | 20.0 | −23.3 |
| (%) | (SD) | (11.57) | (8.96) | (10.23) | (13.14) | (16.58) | (14.77) | (14.77) | (8.67) | (14.22) |
| | median change | 84.0 | 7.0 | 4.0 | 94.0 | 2.0 | −3.0 | 88.0 | −20.0 | −20.0 |
| | (min-max) | (59-111) | (−8-27) | (−21-25) | (76-121) | (−26-50) | (−23-26) | (69-134) | (−34-0) | −60-1) |
| | p-value* | | | | | | 0.5045 | | | <0.0001 |
| Protein C activity | mean change | 95.0 | 1.5 | 1.6 | 96.7 | 10.6 | 9.4 | 97.7 | 17.7 | 18.5 |
| (%) | (SD) | (15.42) | (9.81) | (10.46) | (14.84) | (11.47) | (13.79) | (15.19) | (12.18) | (13.69) |
| | median change | 95.0 | 1.0 | 2.0 | 98.0 | 12 | 8.0 | 97.5 | 19.5 | 18.0 |
| | (min-max) | (73-141) | (−15-32) | (−19-26) | (71-135) | (−11-31) | (−14-36) | (69-122) | (−11-54) | (0-66) |
| | p-value* | | | | | | 0.0794 | | | <0.0001 |
| TFPI | mean change | 1.108 | 0.032 | 0.079 | 0.141 | 0.005 | −0.067 | 0.18 | −0.133 | −0.212 |
| (U/mL) | (SD) | (0.30) | (0.47) | (0.27) | (0.292) | (0.407) | (0.3588) | (0.328) | (0.49) | (0.278) |
| | median change | 1.060 | 0.070 | −0.095 | 1.030 | 0.020 | −0.090 | 1.025 | −0.190 | −0.205 |
| | (min-max) | (0.50-1.60) | (−0.89-0.68) | (0.84-0.55) | (0.69-1.60) | (−0.57-0.80) | (−0.90-0.62) | (0.69-1.60) | (−0.92-0.79) | (−0.67-0.24) |

Fibrinolysis proteins

| | | | | | | | | | | |
|---|---|---|---|---|---|---|---|---|---|---|
| Plasminogen | mean change | 93.5 | 10.2 | 12.2 | 97.8 | 39.3 | 39.1 | 98.2 | 35.2 | 35.5 |
| (%) | (SD) | (10.70) | (8.96) | (9.15) | (13.17) | (10.81) | (13.20) | (11.69) | (14.86) | (12.78) |
| | median change | 94.0 | 12.0 | 12.0 | 96.0 | 39.0 | 39.0 | 98.5 | 32.5 | 37.0 |
| | (min-max) | (68-123) | (−12-29) | (−5-33) | (78-127) | (17-54) | (19-77) | (78-123) | (9-67) | (6-68) |
| | p-value* | | | | | | <0.0001 | | | <0.0001 |
| t-PA | mean change | 4.89 | −0.92 | −0.39 | 5.06 | −2.19 | −2.18 | 5.08 | −1.77 | −2.23 |
| (ng/mL) | (SD) | (2.118) | (1.674) | (1.756) | (2.599) | (2.395) | (2.496) | (2.395) | (1.843) | (1.819) |
| | median change | 4.50 | −0.75 | −0.30 | 4.30 | −1.70 | −1.70 | 4.40 | −1.40 | −1.65 |
| | (min-max) | (2.0-10.6) | (−4.4-2.4) | (−5.5-3.4) | (1.9-12.7) | (−9.3-1.0) | (−9.8-1.0) | (1.5-13.1) | (−5.9-1.4) | (−6.1-1.2) |
| | p-value* | | | | | | 0.0046 | | | 0.0004 |
| PAI-1 | mean change | 1.41 | −0.12 | 0.04 | 1.29 | −0.50 | −0.68 | 1.49 | −0.64 | −0.61 |
| (U/mL) | (SD) | (1.282) | (1.284) | (1.152) | (1.620) | (1.783) | (1.634) | (1.484) | (1.344) | (1.671) |
| | median change | 0.90 | 0.00 | 0.10 | 0.50 | 0.00 | 0.00 | 0.70 | 0.00 | 0.00 |
| | (min-max) | (0.5-5.5) | (−3.5-2.8) | (−2.6-2.1) | (0.5-6.2) | (−5.7-2.0) | (−5.7-1.0) | (0.5-6.2) | (−5.5-0.9) | (−5.2-3.3) |
| | p-value* | | | | | | 0.0751 | | | 0.0816 |

Functional clotting tests

| | | | | | | | | | | |
|---|---|---|---|---|---|---|---|---|---|---|
| ETP-based APC | mean change | 0.881 | 0.089 | 0.076 | 0.745 | 0.555 | 0.624 | 0.724 | 0.734 | 0.864 |
| resistance | (SD) | (0.2184) | (0.2073) | (0.2499) | (0.2294) | (0.2865) | (0.3425) | (0.1494) | (0.2716) | (0.3575) |
| | median change | 0.830 | 0.110 | 0.030 | 0.760 | 0.500 | 0.560 | 0.705 | 0.725 | 0.820 |
| | (min-max) | (0.55-1.63) | (−0.32-0.51) | (−0.61-0.69) | (0.33-1.47) | (0.20-1.47) | (0.06-1.55) | (0.44-1.06) | (0.36-1.37) | (0.34-1.43) |
| | p-value* | | | | | | <0.0001 | | | <0.0001 |
| aPTT-based APC | mean change | 0.49 | 0.06 | 0.08 | 4.47 | −0.00 | 0.43 | 5.08 | −0.15 | −0.02 |
| resistance | (SD) | (0.576) | (0.666) | (0.720) | (1.264) | (0.815) | (0.816) | (0.598) | (0.704) | (0.668) |
| | median change | 4.90 | 0.10 | 0.00 | 4.50 | 0.20 | 0.10 | 5.00 | −0.15 | −0.05 |
| | (min-max) | (3.8-6.3) | (−1.5-1.3) | (−1.1-1.6) | (1.4-6.9) | (−2.1-1.4) | (−0.5-2.4) | (3.9-6.2) | (−2.1-1.3) | (−1.1-1.7) |
| aPTT − APC | mean change | 20.0 | −0.1 | −0.4 | 19.9 | −0.6 | −0.3 | 19.7 | 0.2 | 0.4 |
| (sec) | (SD) | (1.40) | (0.93) | (1.03) | (2.07) | (0.84) | (1.06) | (1.63) | (1.10) | (1.16) |
| | median change | 20.0 | 0.0 | 0.0 | 20.0 | −1.0 | −1.0 | 20.0 | 0.0 | 1.0 |
| | (min-max) | (17-23) | (−2-2) | (−3-1) | (16-25) | (−2-1) | (−2-2) | (17-23) | (−2-2) | (−2-2) |
| | p-value* | | | | | | 0.9836 | | | 0.0329 |
| aPTT + APC | mean change | 99.7 | 0.4 | −0.2 | 89.4 | −3.9 | 6.4 | 99.9 | −1.8 | 2.0 |
| (sec) | (SD) | (16.98) | (16.07) | (15.98) | (31.51) | (19.07) | (19.74) | (17.22) | (16.26) | (15.45) |
| | median change | 98.0 | 0.0 | −1.0 | 86.0 | 0.0 | 2.0 | 97.0 | −0.5 | 2.0 |
| | (min-max) | (69-145) | (−39-29) | (−26-37) | (29-176) | (−56-29) | (−16-54) | (67-130) | (−50-31) | (−22-35) |

Markers of ongoing coagulation

| | | | | | | | | | | |
|---|---|---|---|---|---|---|---|---|---|---|
| Prothrombin | mean change | 0.1374 | 0.0082 | 0.0300 | 0.1265 | 0.0670 | 0.0948 | 0.1260 | 0.0696 | 0.0793 |
| Fragment 1 + 2 | (SD) | (0.0522) | (0.0387) | (0.0506) | (0.0520) | (0.0423) | (0.1272) | (0.0460) | (0.0527) | (0.0484) |
| | median change | 0.1325 | 0.0065 | 0.0290 | 0.1110 | 0.0650 | 0.0640 | 0.1135 | 0.0500 | 0.0745 |
| | (min-max) | (0.060-0.261) | (−0.075-0.095) | (−0.064-0.173) | (0.060-0.332) | (0.003-0.165) | (−0.091-0.639) | (0.073-0.260) | (−0.016-0.201) | (0.001-0.208) |
| | p-value* | | | | | | 0.0021 | | | 0.0005 |
| D-dimer | mean change | 0.300 | 0.014 | 0.039 | 0.439 | −0.027 | −0.065 | 0.351 | 0.021 | 0.024 |
| (μg/mL FEU) | (SD) | (0.0529) | (0.1272) | (0.0866) | (0.4473) | (0.1733) | (0.4206) | (0.1849) | (0.1033) | (0.0934) |
| | median change | 0.270 | 0.000 | 0.010 | 0.270 | 0.000 | 0.020 | 0.270 | 0.000 | 0.000 |
| | (min-max) | (0.27-0.43) | (−0.15-0.59) | (−0.14-0.29) | (0.27-2.28) | (−0.57-0.16) | (−1.92-0.29) | (0.27-1.14) | (−0.27-0.29) | (−0.21-0.25) |

Miscellaneous

| | | | | | | | | | | |
|---|---|---|---|---|---|---|---|---|---|---|
| SHBG | mean change | 68.02 | 37.71 | 39.39 | 73.16 | 46.91 | 47.41 | 73.13 | 183.27 | 187.49 |
| (nmol/L) | (SD) | (21.137) | (29.523) | (31.290) | (28.950) | (35.855) | (36.496) | (23.086) | (51.167) | (59.528) |
| | median change | 64.75 | 30.30 | 30.30 | 67.30 | 51.40 | 49.40 | 70.55 | 173.95 | 204.40 |
| | (min-max) | (25.3-117.9) | (−21.3-102.7) | (−20.4-108.0) | (27.1-144.4) | (−11.8-135.9) | (−24.6-135.1) | (36.2-125.6) | (112.3-301.2) | (89.3-335.4) |
| | p-value* | | | | | | 0.7045 | | | <0.0001 |
| Soluble E-Selectin | mean change | 29.7 | 0.90 | 0.31 | 37.10 | −8.81 | −11.02 | 30.42 | −6.24 | −7.31 |
| (ng/mL) | (SD) | (11.827) | (7.775) | (6.857) | (13.513) | (6.114) | (7.029) | (10.822) | (7.711) | (5.150) |
| | median change | 30.10 | 2.20 | 0.95 | 37.90 | −7.80 | −11.20 | 30.55 | −6.75 | −6.85 |
| | (min-max) | (11.6-58.9) | (−16.0-27.0) | (−10.4-30.4) | (11.3-64.0) | (−23.2-4.6) | (−33.5-2.3) | (13.8-63.4) | (−26.2-14.7) | (−22.1-−0.1) |
| | p-value* | | | | | | <0.0001 | | | <0.0001 |

APC. Activated protein C - TFPI. Free tissue factor pathway inhibitor - t-PA. Tissue type plasminogen inhibitor - PAI-1. Plasminogen activator inhibitor type 1 - SD. standard deviation - SHBG. Sex hormone binding globulin - vWF. von willebrand factor.
*Additional exploratory non-parametric analysis was performed on the absolute change from baseline of the haemostatic, endocrine, liver protein and lipid profile parameters. To explore possible difference between the treatments with regard to the change from baseline at cycle 6, the Kruskal-Wallis test was used. Where a possible difference was detected, pairwise comparisons of the treatments EE/LNG vs. E4/DRSP and EE/DRSP vs. E4/DRSP were done using the Dwass-Steel-Critchlow-Fligner procedure (without adjustment for multiplicity). This test was applied on absolute change from baseline to Cycle 6. In such a case where a possible difference was detected, the corresponding p-value is indicated in the “p-value” line of the corresponding parameter, in the EE/LNG column for a comparison of EE/LNG vs. E4/DRSP and in the EE/DRSP column for a comparison of EE/DRSP vs. E4/DRSP. A p-value ≤ 0.05 indicates that the difference was statistically significant.

TABLE 2
Intraindividual relative change from baseline (hereinafter, “BL”) to Cycles 3 and 6 (%, Mean and Median) (per-protocol population)

| Table 2-Haemostasis parameters. | E4/DRSP | | EE/LNG | | EE/DRSP | |
|---|---|---|---|---|---|---|
| Mean % (+/−SD) | Change BL | Change BL | Change BL | Change BL | Change BL | Change BL |
| Median % | to Cycle 3 | to Cycle 6 | to Cycle 3 | to Cycle 6 | to Cycle 3 | to Cycle 6 |

Coagulation factors

| Fibrinogen | Mean | 10.0 | (21.6) | 9.8 | (18.0) | 15.7 | (28.8) | 13.7 | (31.3) | 21.1 | (24.9) | 18.6 | (22.1) |
|---|---|---|---|---|---|---|---|---|---|---|---|---|---|
| | Median | 3.8 | | 10.2 | | 7.9 | | 4.6 | | 22.0 | | 15.7 | |
| Prothrombin activity | Mean | 6.2 | (8.2) | 6.3 | (9.3) | 13.9 | (9.2) | 11.3 | (9.8) | 14.7 | (9.4) | 11.0 | (12.0) |
| | Median | 9.2 | | 6.6 | | 13.0 | | 13.0 | | 14.4 | | 6.9 | |
| Factor VII activity | Mean | −4.7 | (10.3) | 0.5 | (16.3) | −6.1 | (16.5) | −6.9 | (16.4) | 20.7 | (19.3) | 18.8 | (16.3) |
| | Median | −4.3 | | −3.2 | | −7.2 | | −4.7 | | 18.2 | | 19.8 | |
| Factor VIII activity | Mean | 10.1 | (20.2) | 3.5 | (22.8) | 10.2 | (21.8) | 2.4 | (27.8) | 19.2 | (21.9) | 9.6 | (25.2) |
| | Median | 11.3 | | 4.9 | | 8.6 | | 2.9 | | 20.6 | | 8.6 | |
| vWF | Mean | 6.3 | (14.6) | 7.2 | (18.6) | 0.5 | (17.0) | −1.3 | (18.6) | 11.6 | (15.9) | 13.8 | (18.1) |

| | | | | | | |
|---|---|---|---|---|---|---|
| Median | 5.1 | 4.8 | −1.9 | −2.1 | 7.9 | 13.4 |

Anticoagulant proteins

| Antithrombin | Mean | 0.0 | (5.6) | 0.3 | (5.4) | −2.4 | (6.3) | −3.7 | (6.7) | −1.8 | (7.2) | −1.9 | (8.6) |
|---|---|---|---|---|---|---|---|---|---|---|---|---|---|
| | Median | 0.0 | | −0.9 | | −2.1 | | −5.0 | | −2.3 | | −3.5 | |
| Protein S | Mean | −2.1 | (13.5) | −2.1 | (12.7) | 0.4 | (17.8) | −5.7 | (14.4) | −23.3 | (8.9) | −29.2 | (9.8) |
| | Median | 1.0 | | −4.3 | | −2.4 | | −5.1 | | −26.1 | | −30.6 | |
| Protein S. free | Mean | 4.5 | (10.8) | 4.5 | (11.6) | 3.8 | (19.0) | −0.0 | (15.6) | −21.7 | (8.3) | −24.3 | (11.9) |
| | Median | 7.8 | | 4.9 | | 2.5 | | −3.4 | | −21.1 | | −22.2 | |
| Protein C activity | Mean | 1.9 | (10.2) | 2.0 | (11.0) | 11.9 | (12.8) | 10.7 | (15.0) | 18.8 | (12.5) | 19.4 | (13.9) |
| | Median | 1.2 | | 2.3 | | 12.4 | | 7.1 | | 20 | | 17.8 | |
| TFPI | Mean | −4.7 | (38.8) | −3.7 | (24.2) | 5.7 | (38.6) | −1.7 | (30.2) | −3.3 | (45.5) | −14.2 | (23.3) |

| | | | | | | |
|---|---|---|---|---|---|---|
| Median | 9.0 | −8.4 | −2.6 | −6.2 | −18.9 | −20.4 |

Fibrinolysis proteins

| Plasminogen | Mean | 11.4 | (10.0) | 13.4 | (10.0) | 41.3 | (13.6) | 40.5 | (14.4) | 37.0 | (17.7) | 36.9 | (14.9) |
|---|---|---|---|---|---|---|---|---|---|---|---|---|---|
| | Median | 11.9 | | 12.2 | | 45.3 | | 40.4 | | 32.0 | | 35.3 | |
| t-PA | Mean | −8.4 | (0.3) | −0.0 | (0.3) | −33.7 | (25.9) | −34.5 | (27.1) | −26.7 | (33.7) | −35.6 | (29.9) |
| | Median | −15.7 | | −6.9 | | −34.9 | | −33.3 | | −35.7 | | −39.7 | |
| PAI-1 | Mean | 46.1 | (136.5) | 39.8 | (92.9) | 17.0 | (108.1) | −4.2 | (67.4) | −16.9 | (50.5) | −0.6 | (80.6) |

| | | | | | | |
|---|---|---|---|---|---|---|
| Median | 0.0 | 20.0 | 0.0 | 0.0 | 0.0 | 0.0 |

Functional clotting tests

| ETP-based APC resistance | Mean | 11.0 | (22.5) | 9.9 | (27.9) | 82.1 | (49.8) | 92.0 | (57.9) | 108.8 | (49.5) | 125.1 | (57.5) |
|---|---|---|---|---|---|---|---|---|---|---|---|---|---|
| | Median | 11.9 | | 2.9 | | 69.8 | | 77.9 | | 95.9 | | 121.8 | |
| aPTT-based APC resistance | Mean | 2.0 | (13.2) | 2.8 | (15.3) | 1.8 | (15.5) | 10.9 | (18.5) | −2.3 | (13.3) | 1.1 | (13.5) |
| | Median | 2.1 | | 0.0 | | 4.5 | | 5.0 | | −3 | | −1.0 | |
| aPTT − APC | Mean | −0.4 | (4.6) | −1.7 | (4.9) | −3.0 | (4.2) | −1.1 | (5.5) | 1.4 | (5.6) | 2.0 | (5.9) |
| | Median | 0.0 | | 0.0 | | −4.2 | | −4.0 | | 0.0 | | 4.8 | |
| aPTT + APC | Mean | 1.8 | (15.5) | 1.3 | (16.8) | −1.5 | (16.8) | 9.7 | (23.0) | −0.7 | (15.8) | 3.2 | (16.0) |

| | | | | | | |
|---|---|---|---|---|---|---|
| Median | 0.0 | −1.1 | 0.0 | 4.5 | −0.4 | 2.5 |

Markers of ongoing coagulation

| Prothrombin Fragment 1 + 2 | Mean | 9.6 | (25.4) | 26.2 | (37.5) | 58.8 | (36.4) | 80.0 | (73.7) | 57.6 | (39.1) | 65.5 | (38.4) |
|---|---|---|---|---|---|---|---|---|---|---|---|---|---|
| | Median | 6.7 | | 23.1 | | 62.2 | | 71.1 | | 47.6 | | 64.3 | |
| D-dimer | Mean | 7.8 | (44.1) | 14.3 | (27.8) | 4.7 | (26.2) | 11.0 | (42.7) | 6.4 | (25.9) | 11.5 | (27.7) |

| | | | | | | |
|---|---|---|---|---|---|---|
| Median | 0 | 3.7 | 0.0 | 7.4 | 0.0 | 0.0 |

Miscellaneous

| | | | | | | | | | | | | | |
|---|---|---|---|---|---|---|---|---|---|---|---|---|---|
| SHBG | Mean | 57.9 | (40.67) | 61.3 | (44.23) | 82.4 | (76.92) | 80.0 | (68.3) | 269.3 | (105.3) | 274.7 | (117.6) |

| | | | | | | |
|---|---|---|---|---|---|---|
| Median | 51.4 | 55.1 | 66.9 | 73.8 | 239.5 | 251.5 |

APC. Activated protein C; TFPI. Free tissue factor pathway inhibitor; t-PA. Tissue type plasminogen inhibitor; PAI-1. Plasminogen activator inhibitor type 1; SD. standard deviation; SHBG. Sex hormone binding globulin; vWF. von willebrand factor.

TABLE 3
Mean relative change from baseline to Cycle 3 and 6 (%) (per-protocol)

| Haemostasis parameters. | E4/DRSP | | EE/LNG | | EE/DRSP | |
|---|---|---|---|---|---|---|
| mean (%) | Cycle 3 | Cycle 6 | Cycle 3 | Cycle 6 | Cycle 3 | Cycle 6 |

Coagulation factors

| | | | | | | |
|---|---|---|---|---|---|---|
| Fibrinogen | 4.5 | 8.1 | 10.6 | 10.3 | 18.3 | 16.4 |
| Prothrombin activity | 6.0 | 5.9 | 13.2 | 11.2 | 14.3 | 10.4 |
| Factor VII activity | −5.3 | −1.4 | −7.7 | −8.8 | 20.9 | 18.5 |
| Factor VIII activity | 7.5 | 0.2 | 7.2 | 0.2 | 17.5 | 8.4 |
| vWF | 4.4 | 5.5 | −1.6 | −3.3 | 10.7 | 13.4 |

Anticoagulant proteins

| | | | | | | |
|---|---|---|---|---|---|---|
| Antithrombin | −0.1 | 0.2 | -−2.8 | −4.4 | −2.1 | −2.3 |
| Protein S | 0.6 | −2.5 | −1.2 | −7.0 | −23.7 | −29.6 |
| Protein S. free | 7.6 | 4.4 | 2.4 | −0.2 | −22.0 | −25.3 |
| Protein C activity | 1.6 | 1.7 | 11.0 | 9.7 | 18.1 | 18.7 |
| TFPI | −2.9 | −7.1 | 0.4 | −7.9 | −11.3 | −18.1 |

Fibrinolysis proteins

| | | | | | | |
|---|---|---|---|---|---|---|
| Plasminogen | 10.9 | 13.1 | 40.1 | 40.7 | 35.8 | 36.1 |
| t-PA | −18.8 | −8.0 | −43.2 | −42.9 | −34.8 | −42.5 |
| PAI-1 | −8.5 | 2.8 | −38.5 | −51.3 | −43.3 | −39.8 |

Functional clotting tests

| | | | | | | |
|---|---|---|---|---|---|---|
| ETP-based APC resistance | 10.1 | 8.7 | 74.4 | 81.4 | 102.6 | 120.7 |
| aPTT-based APC | 1.1 | 1.6 | −0.0 | 9.9 | −3.0 | −0.2 |
| resistance | | | | | | |
| aPTT − APC | −0.6 | −1.8 | −3.2 | −1.7 | −1.2 | 1.9 |
| aPTT + APC | −0.4 | −0.2 | −4.4 | 7.0 | −1.8 | 1.5 |

Markers of ongoing coagulation

| | | | | | | |
|---|---|---|---|---|---|---|
| Prothrombin Fragment 1 + 2 | 5.9 | 21.8 | 53.0 | 74.8 | 55.2 | 61.0 |
| D-dimer | 4.7 | 12.9 | −6.1 | −13.6 | 6.1 | 7.7 |

Miscellaneous

| | | | | | | |
|---|---|---|---|---|---|---|
| SHBG | 55.4 | 57.9 | 64.1 | 63.6 | 250.6 | 255.3 |

APC. Activated protein C; TFPI. Free tissue factor pathway inhibitor; t-PA. Tissue type plasminogen inhibitor; PAI-1. Plasminogen activator inhibitor type 1; SHBG. Sex hormone binding globulin; vWF. von willebrand factor.

### Calculation of the VTE Risk

A clinical program parallel to the one reported above assessed the efficacy, cycle control, general safety and acceptability of E4/DRSP in healthy women aged 16-50 years and involved subject participation for 12 months (13 cycles, 1 cycle=28 days). Women with a Body Mass index (BMI) up to 35.0 kg/m² were included in the study. In this clinical trial which involved 3417 subjects, a single case of VTE occurred. Based on the number of subjects in the trial and on the duration of COC administration for each subject, this occurrence can be converted into an estimated VTE risk for the COC of the invention of 3.7 VTE/10 000 women per year.

As reported above, the estimated risk of blood clot occurrence in users of a CHC based on LNG, norethisterone or norgestimate is from 5 to 7 per 10,000 women during one year. For users of a CHC that contains drospirenone, the approximate risk of blood clot occurrence is from 9 to 12 per 10,000 women during one year.

It can thus be seen that the COC of the present invention compares favourably to earlier products of 2^{nd}, 3^{rd} and 4^{th} generation. For reference, the estimated risk of blood clot occurrence in non-CHC users who are not pregnant is around 2 per 10,000 women during one year (in more details, the article by de Bastos et al., “Combined oral contraceptives: venous thrombosis.”; Cochrane Database Syst Rev. 2014 Mar. 3; (3), reports incidences in cohorts of non-CHC users of 1.9 and 3.7 per 10,000 person years, together with earlier findings of 1.6 per 10,000 person years).

From the above it can be concluded that with a VTE occurrence of 3.7 per 10,000 women year, the COC of the present invention is at the upper limit of non-CHC users (3.7 per 10,000 women year reported in Lidegaard et al. (2011) BMJ 343: d6423-see Table 2 on page 10 therein), and far apart from the lower limit of the safest generation of COCs of the prior art (risk of at least 5 per 10,000 women year reported for second generation COCs).

### Second Determination of the ETP-Based APC Resistance

To confirm the data presented above, the inventors undertook a second analysis of the plasma samples collected during the clinical trial described above, and in particular the measurement of the APC resistance based on ETP.

The laboratory protocol for analysing the plasma samples was improved and a new calibration routine was used. The following results were obtained after performing these new measurements, where Table 4 and Table 5 present results with the same formatting as Table 1 and Table 2 above, respectively.

TABLE 4
Change from baseline (hereinafter, “BL”) to Cycles 3 and 6 in levels of Activated Protein C Resistance-ETP based (per-protocol population)

| | E4/DRSP | | | EE/LNG | | | EE/DRSP | | |
|---|---|---|---|---|---|---|---|---|---|
| Table 4 | | Change BL | Change BL | | Change BL | Change BL | | Change BL | Change BL |
| APC-r (ETP-based) | Baseline | to Cycle 3 | to Cycle 6 | Baseline | to Cycle 3 | to Cycle 6 | Baseline | to Cycle 3 | to Cycle 6 |
| mean change +/− | 1.803 | 0.618 | 0.472 | 1.496 | 2.229 | 2.042 | 1.375 | 2.970 | 3.014 |
| SD | (0.7770) | (0.4889) | (0.6360) | (0.9739) | (0.7831) | (0.8805) | (0.5951) | (0.7936) | (0.8506) |
| median change | 1.665 | 0.570 | 0.460 | 1.490 | 2.250 | 1.910 | 1.370 | 2.960 | 3.055 |
| (min-max) | (0.53-3.35) | (−0.47-2.22) | (−0.93-1.80) | (0.00-5.03) | (1.15-3.77) | (0.17-3.43) | (0.46-3.06) | (1.64-4.53) | (1.57-4.58) |
| p-value* | | | | | | <0.0001 | | | <0.0001 |

*Additional exploratory non-parametric analysis was performed on the absolute change from baseline. To explore possible difference between the treatments with regard to the change from baseline at cycle 6, the Kruskal-Wallis test was used. Where a possible difference was detected, pairwise comparisons of the treatments EE/LNG vs. E4/DRSP and EE/DRSP vs. E4/DRSP were done using the Dwass-Steel-Critchlow-Fligner procedure (without adjustment for multiplicity). In such a case, the corresponding p-value is indicated in the “p-value” line, in the EE/LNG column for a comparison of EE/LNG vs. E4/DRSP and in the EE/DRSP column for a comparison of EE/DRSP vs. E4/DRSP. A p-value ≤ 0.05 indicates that the difference was statistically significant.

TABLE 5
Intraindividual relative change from baseline (hereinafter, “BL”) to
Cycles 3 and 6 (%, Mean and Median) (per-protocol population)

| | E4/DRSP | | EE/LNG | | EE/DRSP | |
|---|---|---|---|---|---|---|
| Table 5 | Change BL | Change BL | Change BL | Change BL | Change BL | Change BL |
| APC-r (ETP-based) | to Cycle 3 | to Cycle 6 | to Cycle 3 | to Cycle 6 | to Cycle 3 | to Cycle 6 |
| Mean % | 42.0 | 36.7 | 182.7 | 165.8 | 266.3 | 268.1 |
| (+/−SD) | (34.6) | (52.6) | (114.3) | (98.6) | (162.1) | (170.7) |
| Median % | 39.5 | 30.0 | 165.0 | 164.5 | 229.0 | 218.5 |
| (min-max) | (−19.0-117.0) | (−53.0-233.0) | (33.0-496.0) | (30.0-424.0) | (91.0-781.0) | (99.0-763.0) |

Despite the protocol modifications and the full rerun of all plasma samples, the trend reported in Tables 1-3 above for the ETP-based APC resistance is fully confirmed in Tables 4 and 5 above. In particular, the advantages of the COC of the present invention are directly apparent by comparison with a 4^{th} generation COC (Yaz®), but also, and more importantly, a 2^{nd} generation COC (Melleva®).

Statistical analyses additionally reveal very significant differences from baseline, and especially so for the Yaz® and Melleva® comparator COCs. More importantly, as shown by p-values displayed in Tables 1 and 4, there were very significant differences between the COC of the invention and both comparators, while both comparators were less different to each other.

From the above it appears that the COC of the invention is significantly different from prior art 2^{nd} and 4^{th} generation COCs. Altogether, the measured parameters demonstrate that the haemostatic profile of the COC of the invention is at least comparable to a safest 2^{nd} generation LNG-containing COC, and shows a more favourable effect on haemostasis parameters than a 4^{th} generation DRSP-containing COC.

1. A combined oral contraceptive (COC) method having a reduced risk of venous thromboembolism (VTE) as compared to a COC method comprising administration of drospirenone in combination with ethinylestradiol, comprising orally administering to a female subject an amount of estetrol monohydrate and drospirenone effective to reduce the risk of venous thromboembolism as compared to the risk associated with the use of drospirenone in combination with ethinylestradiol, wherein the estetrol monohydrate is orally administered at a daily dose of 15 mg and the drospirenone is orally administered at a daily dose of from 1 mg to 4 mg, wherein the VTE risk is lower than the VTE risk associated with administration of a combination of 20 μg ethinylestradiol and 3 mg drospirenone.

2-5. (canceled)

6. The method of claim 1, wherein the method is a combined oral administration method with an administration-free interval of about 7 days.

7. The method of claim 1, wherein the method is a combined oral administration method with an administration-free interval of about 4 days.

8. (canceled)

9. (canceled)

10. The method of claim 1, wherein the drospirenone is administered at a daily dose of about 3 mg.

11. The method of claim 1, wherein the estetrol monohydrate and drospirenone are formulated together in a single oral dosage unit.

12. The method of claim 11, wherein the oral dosage unit is formulated to provide a daily dose of the estetrol monohydrate and drospirenone.

13. (canceled)

14. The method of claim 1, wherein the estetrol monohydrate is formulated in a first oral dosage unit and the drospirenone is formulated in a second oral dosage unit.

15. The method of claim 14, wherein the first oral dosage unit is formulated to provide a daily dose of the estetrol monohydrate and the second oral dosage unit is formulated to provide a daily dose of the drospirenone.

16. The method of claim 1, wherein the female subject is selected from first-ever users and switchers/re-starters with a break of more than 4 weeks.

17. A contraceptive method having a reduced risk of venous thromboembolism (VTE) as compared to a method comprising administration of drospirenone in combination with ethinylestradiol, comprising orally administering to a female subject a combined oral contraceptive comprising an amount of estetrol monohydrate and drospirenone effective to reduce the risk of venous thromboembolism as compared to the risk associated with the use of drospirenone in combination with ethinylestradiol, wherein the estetrol monohydrate is administered at a daily dose of 15 mg and the drospirenone is administered at a daily dose of from 1 mg to 4 mg, wherein the VTE risk is lower than the VTE risk associated with administration of a combination of 20 μg ethinylestradiol and 3 mg drospirenone.

18. The method of claim 17, wherein the drospirenone is orally administered at a daily dose of about 3 mg.

19. The method of claim 17, wherein the drospirenone is orally administered at a daily dose of 3 mg.

20. The method of claim 17, wherein the method comprises an administration-free interval of about 7 days.

21. The method of claim 17, wherein the method comprises an administration-free interval of about 4 days.

22. The method of claim 17, wherein the female subject is selected from first-ever users and switchers/re-starters with a break of more than 4 weeks.

23. A method of reducing the risk of venous thromboembolism (VTE) associated with combined oral contraceptive (COC) methods comprising administration of drospirenone, comprising orally administering to a female subject a combined oral contraceptive comprising estetrol monohydrate at a daily dose of 15 mg and drospirenone at a daily dose of from 1 mg to 4 mg, wherein the VTE risk is lower than the VTE risk associated with oral administration of a combination of 20 μg ethinylestradiol and 3 mg drospirenone.

24. The method of claim 23, wherein the method is a combined oral administration method with an administration-free interval of about 4 days or about 7 days.

25. The method of claim 23, wherein the drospirenone is administered at a daily dose of about 3 mg.

26. The method of claim 23, wherein the estetrol monohydrate is formulated in a first oral dosage unit and the drospirenone is formulated in a second oral dosage unit.

27. The method of claim 23, wherein the estetrol monohydrate and drospirenone are formulated together in a single oral dosage unit.
